(19) United States
(12) Patent Application Publication
(10) Pub. No.: US 2020/0181578 A1
(43) Pub. Date: Jun. 11, 2020

(54) MITOCHONDRIAL TRANSPLANTATION TO ALTER ENERGY METABOLISM

(71) Applicant: The Regents of The University of California, Oakland, CA (US)

(72) Inventors: Arash Kheradvar, Irvine, CA (US); Paria Ali Pour, Irvine, CA (US)

(21) Appl. No.: 16/794,115

(22) Filed: Feb. 18, 2020

Related U.S. Application Data
(63) Continuation-in-part of application No. 16/597,498, filed on Oct. 9, 2019.
(60) Provisional application No. 62/743,081, filed on Oct. 9, 2018.

(51) Int. Cl. C12N 5/077; A61K 35/12; A61K 35/34

(52) U.S. Cl. CPC C12N 5/0657; A61K 35/34; A61K 35/12

(57) ABSTRACT

A method of altering energy metabolism in a recipient cell including: identifying the recipient cell as being in need of altering its oxidative phosphorylation status, obtaining exogenous mitochondria, and introducing into the recipient cell the exogenously obtained mitochondria, wherein the exogenously obtained mitochondria functions in the recipient cell to increase or decrease oxidative phosphorylation and/or glycolysis. Also disclosed are isolated cells that include an exogenous mitochondria, wherein the cell demonstrates increased energy metabolism compared to a control cell of the same type but wherein the control cell lacks exogenously added mitochondria. Also disclosed are methods of treating a subject suffering from ischemia or a mitochondrial dysfunction including administering one or more group of isolated cells including exogenous mitochondria as disclosed herein to the subject, wherein the one or more isolated cell including exogenous mitochondria improve symptoms of the ischemia or the mitochondrial dysfunction.

### PRIORITY AND CROSS-REFERENCE TO RELATED APPLICATIONS

This application is a continuation-in-part application of U.S. application Ser. No. 16/597,498, filed Oct. 9, 2019, which claims priority to U.S. Provisional Patent Application No. 62/743,081, filed Oct. 9, 2018; and which claims benefit of U.S. Provisional Application No. 62/935,913, filed Nov. 15, 2019, the entireties of which are hereby incorporated by reference herein.

### STATEMENT REGARDING FEDERALLY SPONSORED R&D

This invention was made with government support under NIH Training Program in Cardiovascular Applied Research and Entrepreneurship (2T32HL116270-06A1). The government has certain rights in the invention.

### FIELD

Some embodiments relate to mitochondrial transplantation into cells, such as cardiomyocytes, skeletal cells, fibroblasts, T cells, B cells or stem cells to alter oxidative phosphorylation and/or glycolysis in a recipient cell.

### BACKGROUND

The mitochondrion found in most eukaryotic cells provides nearly all of the cell's energy by the oxidative phosphorylation process through the mitochondrial respiratory chain (Sebastian D, Palacin M, Zorzano A. Mitochondrial dynamics: Coupling mitochondrial fitness with healthy aging. Trends Mol Med. 2017; 23:201-215; and Svensson O L. Mitochondria : Structure, functions, and dysfunctions. 2010). The eukaryotes' mitochondrion is believed to have evolved from a small, autotrophic bacterium that was engulfed by a larger primitive, heterotrophic, eukaryotic cell (Svensson O L. Mitochondria : Structure, functions, and dysfunctions. 2010). Owing to its origin, mitochondrion has a (maternally inherited) genome that is distinct from the cell's nuclear genome. Although mitochondrion was once a free-living organism, because of its symbiotic relationship through evolution, some of its genome has been translocated to the cell nucleus, making it a semi-autonomous intracellular organelle dependent on the nucleus. Like the nuclear genome, mitochondrial DNA (mtDNA) is constantly prone to damage and mutations. However, mitochondria lack effective DNA repair mechanisms so the mtDNA defects often clonally accumulate in subsequent mitochondria. The majority of mitochondrial diseases occur as a result of mutations in either the nuclear DNA (nDNA) or mtDNA. Regardless, the phenotypic representations of all mitochondrial disorders are deficiencies in energy metabolism and cell function with cardiac involvement as a common manifestation ranging from cardiomyopathy to arrhythmias and heart failure (Svensson O L. Mitochondria : Structure, functions, and dysfunctions. 2010; Schaefer A M, Taylor R W, Turnbull D M, Chinnery P F. The epidemiology of mitochondrial disorders—past, present and future. Biochim Biophys Acta. 2004; 1659:115-120; and El-Hattab A W, Scaglia F. Mitochondrial cardiomyopathies. Front Cardiovasc Med. 2016; 3:25).

Recently, much interest has been devoted to cellular biotherapies involving mitochondria. Elliot et al. have successfully demonstrated that the introduction of normal mitochondria into human breast cancer cells restores mitochondrial function, inhibits cancer cell proliferation, and reverses chemoresistance by increasing the sensitivity of cells to breast cancer medication (Elliott R L, Jiang X P, Head J F. Mitochondria organelle transplantation: Introduction of normal epithelial mitochondria into human cancer cells inhibits proliferation and increases drug sensitivity. Breast Cancer Research and Treatment. 2012; 136:347-354). Moreover, early clinical feasibility of mitochondrial replacement therapy (MRT) in human has provided hope to mitigating inherited mitochondrial disorders (Zhang J, Liu H, Luo S, Lu Z, Chavez-Badiola A, Liu Z, Yang M, Merhi Z, Silber S J, Munne S, Konstantinidis M, Wells D, Tang J J, Huang T. Live birth derived from oocyte spindle transfer to prevent mitochondrial disease. Reprod Biomed Online. 2017; 34:361-368). and transplantation of mitochondria into the ischemic zone of myocardium has been shown to improve recovery in cardiac ischemia-reperfusion injury (Moskowitzova K, Shin B, Liu K, Ramirez-Barbieri G, Guariento A, Blitzer D, Thedsanamoorthy J K, Yao R, Snay E R, Inkster J A H, Orfany A, Zurakowski D, Cowan D B, Packard A B, Visner G A, Del Nido P J, McCully J D. Mitochondrial transplantation prolongs cold ischemia time in murine heart transplantation. J Heart Lung Transplant. 2019; 38:92-99; Emani S M, McCully J D. Mitochondrial transplantation: Applications for pediatric patients with congenital heart disease. Transl Pediatr. 2018; 7:169-175; Ramirez-Barbieri G, Moskowitzova K, Shin B, Blitzer D, Orfany A, Guariento A, Iken K, Friehs I, Zurakowski D, Del Nido P J, McCully J D. Alloreactivity and allorecognition of syngeneic and allogeneic mitochondria. Mitochondrion. 2018; Cowan DB, Yao R, Thedsanamoorthy J K, Zurakowski D, Del Nido P J, McCully J D. Transit and integration of extracellular mitochondria in human heart cells. Sci Rep. 2017; 7:17450; Shin B, Cowan D B, Emani S M, Del Nido P J, McCully J D. Mitochondrial transplantation in myocardial ischemia and reperfusion injury. Adv Exp Med Biol. 2017; 982:595-619; McCully J D, Cowan D B, Emani S M, Del Nido P J. Mitochondrial transplantation: From animal models to clinical use in humans. Mitochondrion. 2017; 34:127-134; Emani S M, Piekarski B L, Harrild D, del Nido P J, McCully J D. Autologous mitochondrial transplantation for dysfunction after ischemia-reperfusion injury. The Journal of Thoracic and Cardiovascular Surgery. 2017; 154:286-289; Kaza A K, Wamala I, Friehs I, Kuebler J D, Rathod R H, Berra I, Ericsson M, Yao R, Thedsanamoorthy J K, Zurakowski D, Levitsky S, Del Nido P J, Cowan D B, McCully J D. Myocardial rescue with autologous mitochondrial transplantation in a porcine model of ischemia/reperfusion. J Thorac Cardiovasc Surg. 2017; 153:934-943; Cowan D B, Yao R, Akurathi V, Snay E R, Thedsanamoorthy J K, Zurakowski D, Ericsson M, Friehs I, Wu Y, Levitsky S, Del Nido P J, Packard A B, McCully J D. Intracoronary delivery of mitochondria to the ischemic heart for cardioprotection. PLoS One. 2016; 11:e0160889; McCully J D, Levitsky S, del Nido P J, Cowan D B. Mitochondrial transplantation for therapeutic use. Clinical and Translational Medicine. 2016; 5:16; and Pacak C A, Preble J M, Kondo H, Seibel P, Levitsky S, del Nido P J, Cowan D B, McCully J D. Actin-dependent mitochondrial internalization in cardiomyocytes: Evidence for rescue of mitochondrial function. Biology Open. 2015; 4:622). The mechanism of mitochondrial uptake is a question that is actively under investigation and some of the suggested mechanisms are actin-dependent endocytosis (Pacak C A, Preble J M, Kondo H, Seibel P, Levitsky S, del Nido P J, Cowan D B, McCully J D. Actin-dependent mitochondrial internalization in cardiomyocytes: Evidence for rescue of mitochondrial function. Biology Open. 2015; 4:622) and macropinocytosis (Kitani T, Kami D, Matoba S, Gojo S. Internalization of isolated functional mitochondria: Involvement of macropinocytosis. Journal of Cellular and Molecular Medicine. 2014; 18:1694-1703). However, little is known about the intracellular fate of mitochondria during transplantation and in particular the bioenergetic consequences of mitochondrial transplantation. We have addressed this question by studying how cellular bioenergetics are affected in the short- and long-term after mitochondrial transplantation.

Mitochondrial cardiomyopathy is a condition in which the heart-muscle structure, function, or both could be abnormal due to genetic defects involving the mitochondrial respiratory chain. There is currently no treatment for mitochondrial cardiomyopathy and medication is limited to use of various dietary supplements.

### SUMMARY

Disclosed herein are methods of controlling energetics in cells by transplanting mitochondria into the cells. A variety of cells may be modified by mitochondrial transplantation, including but not limited to cardiomyocytes, T cells, B cells and stem cells. In some embodiments, the performance of cells with mitochondrial defects can be enhanced by transplanting normal or non-defective mitochondria (mitochondria carrying no/low mutation load). In other embodiments, defective cells (e.g., cancer cells) may be impaired, potentially lethally, by introducing defective mitochondria. Changes in oxidative phosphorylation, mediated by transplanting mitochondria into cells, can modulate acute effects of mitochondrial disorders that cause deficiencies in energy metabolism and cell function. For example, treatment of cardiomyocytes may compensate for diseases ranging from cardiomyopathy to arrhythmias and heart failure.

Some embodiments relate to a method of altering energy metabolism in a recipient cell including:

identifying the recipient cell as being in need of altering its oxidative phosphorylation status,

obtaining exogenous mitochondria, and

introducing into the recipient cell the exogenously obtained mitochondria,

wherein the exogenously obtained mitochondria functions in the recipient cell to increase or decrease oxidative phosphorylation and/or glycolysis.

In some examples, the exogenously obtained mitochondria are autologous compared to the recipient cell.

In some examples, the exogenously obtained mitochondria are non-autologous compared to the recipient cell.

In some examples, the exogenously obtained mitochondria are obtained from a xenogeneic source.

In some examples, the recipient cell was previously subjected to ischemia or is in an individual suffering from a mitochondrial dysfunction.

In some examples, the mitochondrial dysfunction is a condition selected from the group consisting of diabetes; a neurodegenerative disease, a neuromuscular disease, a metabolic disease, Huntington's disease; cancer; Alzheimer's disease; Parkinson's disease; amyotrophic lateral sclerosis; bipolar disorder; schizophrenia; aging; senescence; an anxiety disorder; a cardiovascular disease; sarcopenia; chronic fatigue syndrome; Leigh syndrome; Mitochondrial myopathy; Leber's hereditary optic neuropathy; Mitochondrial DNA depletion syndrome; Myoneurogenic gastrointestinal encephalopathy; and Mitochondrial Encephalopathy, Lactic acidosis, and Stroke-like episodes (MELAS) syndrome.

In some examples, the recipient cell is located within a tissue in a subject.

In some examples, the tissue is myocardium and the recipient cell is a cardiomyocyte.

In some examples, the recipient cell is an immune cell.

In some examples, the immune cell is selected from the group consisting of a T-cell, a B-cell, a monocyte and a natural killer (NK) cell.

In some examples, an increase in oxidative phosphorylation is accompanied by one or more of increased basal respiration, increased maximal respiration, increased coupling efficiency, reduced reactive oxygen species generation, enhanced ATP production, increased spare respiration capacity, and reduced proton leak.

Some embodiments relate to a method of killing a recipient cell or diminishing oxidative phosphorylation and/or glycolysis in a recipient cell including:

obtaining an exogenous mitochondria that is defective or less efficient with regard to energy metabolism, and

introducing into the recipient cell the exogenously obtained mitochondria,

wherein the exogenously obtained mitochondria functions in the recipient cell to reduce energy metabolism.

In some examples, the diminished energy metabolism is accompanied by one or more of reduced basal respiration, reduced maximal respiration, reduced coupling efficiency, increased reactive oxygen species generation, reduced ATP production, decreased spare respiration capacity, and increased proton leak.

In some examples, the recipient cell is neoplastic.

Some embodiments relate to an isolated cell that includes an exogenous mitochondria, wherein the cell demonstrates increased energy metabolism compared to a control cell of the same type but wherein the control cell lacks exogenously added mitochondria.

In some examples, the cell is selected from the group consisting of a muscle cell, an immune cell, a stem cell, and a progenitor cell.

In some examples, the stem cell is a hematopoietic stem cell (HSC).

In some examples, the HSC is a bone marrow-derived mononuclear cell (BMNC).

In some examples, the immune cell is selected from the group consisting of a T cell, a B cell, a monocyte and an NK cell.

In some examples, the exogenous mitochondria is autologous relative to the isolated cell.

In some examples, the exogenous mitochondria is non-autologous relative to the isolated cell.

In some examples, the exogenous mitochondria is derived from a cell of a species that is different compared to the isolated cell.

Some embodiments relate to a method of treating a subject suffering from ischemia or a mitochondrial dysfunction including administering one or more group of isolated cells including exogenous mitochondria as disclosed herein to the subject, wherein the one or more isolated cell including exogenous mitochondria improve symptoms of the ischemia or the mitochondrial dysfunction.

In some examples, the subject is suffering from myocardial ischemia and reduced mitochondrial function, and wherein the administered cells improve or restore normal mitochondrial function in myocardium in the subject.

Some aspects relate to a method of enhancing the cellular activity of a recipient group of cells including:

identifying at least one recipient cell;

obtaining at least one mitochondrion from a donor;

contacting, directly or indirectly, the donor mitochondria with the recipient cell(s) for a period of time sufficient for the recipient cell to uptake the mitochondria, thereby generating a modified recipient cell;

wherein the modified recipient cell exhibits improved energy metabolism represented by one or more of the following characteristics:
(i) increased basal respiration,
(ii) increased maximal respiration,
(iii) increased coupling efficiency,
(iv) reduced reactive oxygen species generation,
(v) enhanced ATP production efficiency,
(vi) increased spare respiration capacity, and
(vii) reduced proton leak, and
wherein the modified recipient cell exhibiting one or more of said characteristics is indicative of enhanced cellular activity.

In some examples, the contacting occurs through the co-incubation of the cells with mitochondria.

In some examples, the contacting occurs through injection of the mitochondria into the recipient cells.

In some examples, the contacting is indirect through stem cells or nanoparticles.

In some examples, the contacting is indirect and includes co-culturing the at least one recipient cell with the at least one donor mitochondria in vitro.

In some examples, the donor mitochondria is obtained from a skeletal muscle cell, and wherein the at least one recipient cell is a cardiomyocyte.

In some examples, the contacting is direct and includes delivery of the donor mitochondria to a recipient cellular tissue space.

In some examples, the recipient cellular tissue space is selected from the group consisting of bone marrow, cardiac tissue, skeletal muscle, liver tissue, the pancreas, the thyroid gland, and the adrenal gland.

In some examples, the recipient cell is an immune cell or a stem cell.

In some examples, the recipient cell is autologous with respect to the donor.

In some examples, the recipient cell is allogeneic with respect to the donor.

In some examples, the recipient cell is xenogeneic with respect to the donor.

Some aspects relate to a method of for reducing or eliminating the cellular activity of a recipient cell including:

identifying at least one recipient cell;

obtaining at least one mitochondrion from a donor, wherein the mitochondria include at least a partial defect in mitochondrial function;

contacting, directly or indirectly, the donor mitochondria with the recipient cell for a period of time sufficient for the recipient cell to uptake the mitochondria, thereby generating a modified recipient cell;

wherein the modified recipient cell exhibits one or more of the following characteristics:
(i) decreased basal respiration,
(ii) decreased maximal respiration,
(iii) decreased coupling efficiency,
(iv) increased reactive oxygen species generation,
(v) reduced ATP production efficiency,
(vi) reduced spare respiration capacity, and
(vii) increased proton leak, and
(viii) Apoptosis, and

wherein the modified recipient cell exhibiting one or more of said characteristics is indicative of reduced cellular activity.

In some examples, the recipient cell is a neoplastic cell, an immune cell or a stem cell.

In some examples, the recipient cell is autologous with respect to the donor.

In some examples, the recipient cell is allogeneic with respect to the donor.

In some examples, the recipient cell is xenogeneic with respect to the donor.

### BRIEF DESCRIPTION OF THE DRAWINGS

FIG. 1. Mitochondrial transplantation studies at a glance. Feasibility of mitochondrial transplantation (autologous, non-autologous, and interspecies) was demonstrated using (a) widefield time-lapse, (b) confocal and (c) holotomography microscopy. The bioenergetics consequences of non-autologous mitochondrial transplantation were studied using a Seahorse Flux Analyzer (a) 2-day, (b) 7-day, (c) 14-day, and (d) 28-day post transplantation. Mitochondrial superoxide was measured using MitoSOX Red Mitochondrial Superoxide Indicator (Mito-HE).

FIG. 2. Internalization of mitochondria into the H9c2 cardiomyocyte-like cells through co-incubation. A. Feasibility of mitochondrial transplantation. The newly internalized mitochondria labeled with pHrodo Red SE (A.1.); the cell's native mitochondria labeled with MitoTracker Green FM (A.2.); the nucleus is labeled with NucBlue Live. The image was taken after the 28-hour time-lapse structured illumination epifluorescence microscopy (Keyence, Itasca, Ill.). B. Dynamics of mitochondrial internalization. Hour 5:00 represent the cardiomyocytes before mitochondrial internalization. The media contains the fluorescently-labeled mitochondria. Since pHrodo Red SE fluoresces brightly red only after it has been internalized by the cell, no florescence could yet be observed. At 5:05, the cardiomyocyte starts taking in the mitochondria, as indicated by the red fluorescence signal (black arrow). As time passes, more mitochondria are internalized into the cell. Moreover, there are interactions between the mitochondria, which are assumed to represent the dynamics of fusion and fission processes (Olympus, Tokyo, Japan). C. Non-autologous transplantation of mitochondria. Mitochondria from rat L6 skeletal muscle cell (shown in red) were transplanted into H9c2 cardiomyocyte (Nanolive S A, Ecublens, Switzerland). D. Interspecies transplantation of mitochondria. Mitochondria from rat L6 skeletal muscle cell (shown in red) were transplanted into the human ARPE-19 retinal epithelial cells (Carl Zeiss AG, Oberkochen, Germany).

FIG. 3. Non-autologous mitochondrial transplantation of L6 skeletal cells into H9c2 cardiomyocytes leads to enhanced bioenergetics 2-day post-transplantation. Top. Oxygen Consumption Rate (OCR) and Extracellular Acidification Rate (ECAR) kinetics increase 2-day post-transplantation indicating the improved metabolism after mitochondrial transplantation. Abbreviations: Oligo=Oligomycin; FCCP: carbonylcyanide-p-trifluoromethoxyphenyl hydrazine; R+A=Rotenone and Antimycin A. Bottom. Compared to control (lavender), there is a statistically-significant increase in non-mitochondrial respiration, ATP production, and basal respiration in the transplant (purple) group. Maximal respiration and spare respiratory capacity are also enhanced, although not statistically significantly. Coupling efficiency and proton leak remain unchanged. An unpaired two-tailed unequal variance student t-test was used and results with p<0.05 were considered as statistically significant (n=28 for transplant and n=12 for control).

FIG. 4. The acute enhancement in bioenergetics after mitochondrial transplantation diminishes in long-term. Top. No significant change in OCR and ECAR kinetics can be observed 7-day, 14-day and 28-day post-transplantation. Abbreviations: Oligo=Oligomycin; FCCP: carbonylcyanide-p-trifluoromethoxyphenyl hydrazine; R+A=Rotenone and Antimycin A. Bottom. No significant difference is found between the transplant (lavender) and control (purple) groups in long-term. While the bioenergetics return to the base-line levels in long-term, no negative consequences to the cell's bioenergetics was also observed due to the intervention. An unpaired two-tailed unequal variance student t-test was used and results with p<0.05 were considered as statistically significant (n=28 for transplant and n=12 for control for t=7-day and 14-day, n=27 for transplant (due to well-measurement failure in one well) and n=12 for control for t=28-day).

FIG. 5. Mitochondrial superoxide production. 48-hour post mitochondrial transplantation, H9C2 cells were exposed to MitoSOX Red reagent to measure superoxide production. MitoSOX Red was excited at 485 nm and fluorescence emission was measured at 590 nm. The whisker box plot shows the distribution of the fluorescence readings for 10 measurements per group. Symbols: white line denotes the population median, “x” indicates the population mean, and filled black dots represent outliers. No statistically-significant difference was observed in the population's mean of the post-transplant groups vs. control. The fluorescence measurements of MitoSOX-treated groups were normalized to the untreated control and displayed as bar graphs, for the mean±SD.

FIG. 6. Effect of high passage number (aging) on mitochondrial function. Mitochondrial function was reduced with increasing passage number. This is indicated by the decrease in ATP production and coupling efficiency—positive indicators of mitochondrial function—and increase in proton leak—a negative indicator of mitochondrial function. An unpaired two-tailed unequal variance student t-test was used and results with p <0.05 were considered as statistically significant (n=28 for p# 12, 14, and 17 and n=27 for p# 23 for transplant and n=12 for p# 12, 14, 17, and 23 for control).

FIG. 7. Non-autologous Transplantation of dermal fibroblast Mitochondria into Jurkat cells (immortalized T cells) are shown using live cell imaging. pHrodo Red representative of transplanted mitochondria excited by 561 nm laser and fluorescence detected for 561-642 nm range. (A) control Jurkat cells; (B) Post-transplant Jurkat cells.

FIG. 8. Validation of the active internalization of mitochondria. To investigate whether the pHrodo dye by itself can get passively internalized into the cell or not. (A) pHrodo Red alone & no cells. (B) cells only & no dye. (C) cells & pHrodo Red. (D) cells, native mitochondria labeled w/MitoTracker Green FM & pHrodo Red. All four groups lacked isolated mitochondria. No signal was detected in the red channel, indicating that the pHrodo dye cannot get passively internalized into the cell.

FIG. 9. (A)-(G) depict a schematic of mitochondrial transplantation. (A) shows cardiomyocyte cells, H9c2 derived from rat embryonic hearts (or L6 rat skeletal muscle cell). (B) shows mitochondrial isolation from cultured cells. (C) shows the native cell's mitochondria are labeled with MitoTracker Green FM. (D) shows isolated mitochondria are labeled with pHrodo Red SE. (E) shows the isolated-labeled mitochondria are co-incubated with cardiomyocytes. (F) shows dynamic behavior of mitochondrial internalization is captured using time-lapse microscopy. (G) shows additional mitochondria are internalized into cardiomyocytes.

FIG. 10. Schematic of the studies on the bioenergetic consequences of mitochondrial transplantation.

FIG. 11. (A)-(D) show mitochondrial transplantation and resultant functional gain. The experiment relates to non-autologous interspecies mitochondrial transplantation of rat L6 skeletal cells in ARPE-19 human retinal cells and the resultant functional gain in Rho Zero Mitochondrial-depleted cells. (A) shows ARP-19 WT cells, no transplantation. (B) shows ARPE-19 Rho Zero cells post-mitochondrial transplantation in WT media. (C) shows ARPE-19 Rho Zero cells post-transplantation initially in Rho zero media and moved to WT media (after 24 hours and for the second 24-hour period). (D) shows ARPE-19 Rho zero cells.

FIG. 12 relates to the bioenergetics of mitochondrial transplantation in ARPE-19 Rho Zero Mitochondrial-depleted Cells. Almost two-fold increase post-mitochondrial transplantation (mitochondria from five million ARPE-19 WT cells into 50K ARPE-19 rho zero cells) in basal respiration of rho zero cells. (i): ARPE-19 WT, (ii): ARPE-Rho Zero Cell Post-mitochondrial Transplantation (media included uridine supplementation at the time of transplantation and was changed to WT media after 24 hours), (iii): ARPE-19 Rho Zero Cell Post-mitochondrial Transplantation (changed to WT media at the time of transplantation), (iv): ARPE-19 Rho Zero Cell (Rho zero media with uridine supplement), (v): ARPE-19 Rho Zero Cell (WT media). (n=4).

FIG. 13 shows cardiomyocyte cell seeding density optimization (40K cells).

FIG. 14 shows cardiomyocyte optimization of FCCP concentration is (1 μM (n=5)).

FIG. 15 shows data related to internalization of mitochondria in cardiomyocytes and possible fission of mitochondria, resulting in two signals emerging from one.

### DETAILED DESCRIPTION

Some embodiments are related to using mitochondrial transplantation to (1) positively modulate cellular bioenergetics to improve or enhance cellular bioenergetics and function post-mitochondrial transplantation with the intent of improving cellular performance or restoring cellular homeostasis (2) negatively modulate cellular bioenergetics to target diseased cells or neoplastic cells for destruction

It is anticipated that transplantation of mitochondria either directly or in conjunction with stem cells can reduce the mitochondrial mutation load in mitochondrial diseases.

It is anticipated that transplantation of mitochondria either directly or in conjunction with stem cells can mitigate other complex diseases in which the mitochondria is dysfunctional, such as but not limited to cardiovascular diseases, neurodegenerative diseases such as but not limited to Parkinson's, Alzheimer's, neuromuscular diseases, retinal diseases such as but not limited to retinitis pigmentosa, cancer, and aging.

It is anticipated that transplantation of mitochondria into immune cells such as but not limited to T cells, can enhance the performance of the immune system in attacking and destroying diseased cells such as but not limited to cancerous cells.

In other settings, it is anticipated that transplantation of defective mitochondria into diseased cells such as but not limited to cancerous cells may impair the function of these cells, potentially lethally, by making them more prone to attacks by the immune cells or more susceptible to cellular autophagy.

According to the endosymbiosis theory of mitochondrial origin, the mitochondrion was once an aerobic prokaryote, more specifically an alpha-proteobacteria that was engulfed by our ancestral eukaryote through an endosome. This is also the origin for mitochondria' sdouble outer membrane. Although mitochondria is semi-autonomous now, it was once free living. Thus, based on the endosymbiosis theory of mitochondrial origin, we hypothesized and tested the feasibility of mitochondrial transplantation.

Mitochondrial transplantation is beneficial in cases of mitochondrial cardiomyopathy, which is a condition in which the heart-muscle structure, function, or both could be abnormal. This is due to genetic defects involving the mitochondrial respiratory chain, in the absence of other concomitant heart disease. More broadly mitochondrial transplantation may be beneficial in mitigating conditions in which the mitochondria is dysfunctional. Unfortunately, there is no therapy for mitochondrial diseases. Patients are given a mitochondrial cocktail that usually only has minor positive effect in managing the condition. In some embodiments, the goal is to test whether mitochondrial transplantation would be a viable cellular biotherapy in helping patients suffering from a mitochondrial disease. In other embodiments, the goal is to test whether mitochondrial transplantation would be an effective cellular biotherapy for other diseases where the mitochondria is dysfunctional.

To test for the feasibility of mitochondrial transplantation, isolated mitochondria were labeled with pHrodo Red (or pHrodo Green STP ester), which is unique in that it is pH dependent and only fluoresces brightly red after a pH drop. Thus, the lack of fluorescence outside the cell served as a clear indication of transplantation's success when a signal is seen inside the cell.

Mitochondria were isolated from 20 million mitochondrial-donor cells (cardiomyocytes, skeletal cells, retinal epithelial cells, fibroblasts, Jurkats—immortalized T cells) using a commercially available mitochondrial isolation kit. The cell's native mitochondria were labeled with MitoTracker Green FM and the isolated mitochondria were labeled with pHrodo Red Succnimidyl Ester. In some embodiments, the mitochondria were labeled for about 10 min to about 1 hour. In some embodiments, the mitochondria were labeled for about 20 min to about 2 hours. In some embodiments, the mitochondria were labeled for about 40 min to about 4 hours. In some embodiments, the mitochondria were labeled for about 80 min to about 8 hours. Two wash steps was performed to remove the excess MitoTracker Green FM dyes and two wash steps to remove the excess pHrodo Red SE dyes. The fluorescently-labeled-isolated mitochondria were co-incubated with the recipient cells. Dynamics of internalization under a fluorescence microscope in a 28-hour time-lapse study was assessed and the respective data was analyzed using ImageJ and IMARIS softwares.

We demonstrate that autologous, non-autologous, and interspecies mitochondrial transplantation into different rat and human cell lines is feasible.

Control experiments were performed to ensure that mitochondria were being actively endocytosed instead of the dye being passively internalized. We tested whether the pHrodo Red dye by itself can get internalized into the cell to rule out the possibility of signal originating from the passive internalization of pHrodo Red. This experiment was performed in replicates and the four groups included were: Group I—pHrodo Red only, with no cells; Group II—Cells only, with no dye; Group III—Cells and pHrodo Red; and Group IV—-Cells with both pHrodo Red and Mito Tracker Green FM.

With Group I, no signal was observed as there were no cells. In the second group with cells only, the cells replicate and remain alive for the course of the 28-hour study. For Groups III and IV, no passive internalization of the dye was detected, as there was no red fluorescence. While the cells in group III and IV appeared to die after about seven to eight hours post co-incubation (most likely due to the toxicity of the high concentration of the dye available in the media), we concluded that the dye did not passively get internalized into the cell, since mitochondrial internalization was observed in a period as early as about 4hours.

In some embodiments, the mitochondria were internalized by the cell in less than 1 hour, after about 1, 2, 3, 4, 5, 6, 7, 8, 9, 10, 11, 12, 13, 14, 15, 16, 17, 18, 19, 20, 21, 22, 23, or 24 hours, or a value within a range defined by any two of the aforementioned values. In some embodiments, the mitochondria were internalized by the cell after about 4, 8, 12, 16, 20, 24, 28, 32, 36, 40, 44, 48, 52, 56, or 60 hours, or a value within a range defined by any two of the aforementioned values. In some embodiments, two signals were obtained from one internalized mitochondrial signal. Without being limited by any particular theory, it is believed that this phenomenon represents fission, which is when mitochondria undergo a decrease in mitochondrial mass and an increase in mitochondrial number. In some embodiments, this may be due to fragmentation.

“Mitochondrial fission” is the division of a single mitochondrial organelle into two or more independent mitochondrial structures.

“Mitochondrial fusion” occurs when two or more mitochondria fuse together into a single mitochondrial structure.

When cells experience metabolic or environmental stresses, mitochondrial fusion and fission work to maintain functional mitochondria. An increase in fusion activity leads to mitochondrial elongation, whereas an increase in fission activity results in mitochondrial fragmentation. The components of this process can influence programmed cell death and lead to cellular dysfunction. Such cell death can be caused by disruptions in the process of either fusion or fission or in combination with disruption of both fission and fusion and mitophagy [0057] The number and shapes of mitochondria in cells are continually changing via a combination of fission and fusion biogenesis and mitophagy. Specifically, fusion assists in modifying stress by integrating the contents of slightly damaged mitochondria as a form of complementation. By enabling genetic complementation, fusion of the mitochondria allows for two mitochondrial genomes with different defects within the same organelle to individually encode what the other lacks. In doing so, these mitochondrial genomes generate all of the necessary components for a functional mitochondrion.

Fission is a process in which mitochondria undergo an overall increase in number and decrease in mass. In some embodiments, the fission process results in about 2-fold to about 20-fold increase in number and about 2-fold to about 20-fold decrease in mass. In some embodiments, the fission process results in about 5-fold to about 10-fold increase in number and about 5-fold to about 10-fold decrease in mass. In some embodiments, the fission process results in about >1-fold to about 5-fold increase in number and about >1-fold to about 20-fold decrease in mass.

We demonstrate that mitochondria can be actively endocytosed into cells. This phenomenon was validated by showing that the pHrodo dye by itself cannot get passively internalized. In some embodiments, we demonstrate that mitochondria can be transplanted into cardiomyocyte through co-incubation.

In some embodiments, whether the transplanted mitochondria are naturally adopted by the host cardiomyocyte can be determined via analyzing the mt-DNA copy number, fission/fusion dynamics, mitophagy, and/or a combination thereof. It is anticipated that by introducing new mitochondria into the host, the host adopts the new mt-DNA and the ratio of the internalized to host's mt-DNA increases overtime, which can be quantitatively evaluated by evaluating the internalized and host's mt-DNA copy number, fission/fusion dynamics, mitophagy, and through a time-course study of mitochondrial genome sequencing pre and post-transplantation.

In some embodiments, the native cell to be transplanted can be muscle cells (e.g., a cardiomyocytes, skeletal muscle cells), blood cells (e.g., T-cells and B-cells), fibroblasts, stem cells, and others.

In some embodiments, testing whether mitochondrial transplantation can lead to improved bioenergetics and function is disclosed. In some embodiments, the use of Seahorse Extracellular Flux analysis is disclosed to determine whether mitochondrial transplantation in normal cardiomyocytes leads to improved bioenergetics and function by measuring oxygen consumption rate (OCR) and extracellular acidification rate (ECAR).

In some embodiments, the source of mitochondria can be any eukaryotic cell. In some embodiments, source of mitochondria can be human, rat, mouse, guinea pig, pig, cattle, chicken, dog, cat, and the like. In some embodiments, the mitochondria can be transplanted into any eukaryotic cell. In some embodiments, the mitochondria can be transplanted into any type of cell from human, rat, mouse, guinea pig, pig, cattle, chicken, dog, cat, and the like. In some embodiments, the mitochondria can be transplanted into cardiomyocytes, retinal cells, muscle cells, adipocytes, hepatocytes, immune cells, neurons, nephrocytes, etc. from human, rat, mouse, guinea pig, pig, cattle, chicken, dog, cat, and the like.

In some embodiments, mitochondrial transplantation results in an increase in the basal respiration of the transplanted. In some embodiments, a change in cellular bioenergetics is determined by evaluating the oxygen consumption rate (OCR) of cells. In some embodiments, a change in cellular bioenergetics is determined by evaluating the Extracellular Acidification Rate (ECAR). In some embodiments, a change in cellular bioenergetics is determined by evaluating the oxygen consumption rate (OCR) of cells, and the Extracellular Acidification Rate (ECAR).

In some embodiments, mitochondrial transplantation results in an increase in the bioenergetic indices such as but not limited to basal respiration of the transplanted cell. In some embodiments, mitochondrial transplantation results in a 1.5-fold, 2-fold, 2.5-fold, 3-fold, 3.5-fold, 4-fold, a 4.5-fold, 5-fold, 5.5-fold, 6-fold, 6.5-fold, 7-fold, 7.5-fold, 8-fold, 8.5-fold, 9-fold, 9.5-fold, 10-fold, or a 20-fold increase in bioenergetic indices such as but not limited to basal respiration of the transplanted cell. In some embodiments, mitochondrial transplantation results in >1 fold to about 5 fold increase in the one bioenergetic index or more indices of the transplanted cell. In some embodiments, mitochondrial transplantation results in >1 fold to about 10 fold increase in one bioenergetic index or more indices of the transplanted cell. In some embodiments, mitochondrial transplantation results in >1 fold to about 20 fold increase in one bioenergetic index or more indices of the transplanted cell.

In some embodiments, mitochondrial transplantation results in decrease in the bioenergetic indices such as but not limited to basal respiration of the transplanted cell. In some embodiments, mitochondrial transplantation results in a 1.5-fold, 2-fold, 2.5-fold, 3-fold, 3.5-fold, 4-fold, a 4.5-fold, 5-fold, 5.5-fold, 6-fold, 6.5-fold, 7-fold, 7.5-fold, 8-fold, 8.5-fold, 9-fold, 9.5-fold, 10-fold, or a 20-fold decrease in bioenergetic indices such as but not limited to basal respiration of the transplanted cell. In some embodiments, mitochondrial transplantation results in >1 fold to about 5 fold decrease in the one bioenergetic index or more indices of the transplanted cell. In some embodiments, mitochondrial transplantation results in >1 fold to about 10 fold decrease in one bioenergetic index or more indices of the transplanted cell. In some embodiments, mitochondrial transplantation results in >1 fold to about 20 fold decrease in one bioenergetic index or more indices of the transplanted cell.

In some embodiments, the bioenergetics of the transplanted mitochondria as measured by OCR, ECAR, or both, can be calculated over the short term, medium term, long term, or a combination thereof.

In some embodiments, the short term refers to about 24 hours to about 48 hours post mitochondrial transplantation. In some embodiments, the medium term refers to about >2 days to about <7 days post mitochondrial transplantation. In some embodiments, the medium term refers to about >7 days post mitochondrial transplantation.

In some embodiments, the bioenergetics of a cell transplanted with mitochondria can increase or decrease over the short term as compared to the medium term, long term, or both.

In some embodiments, the bioenergetics of a cell transplanted with mitochondria can increase or decrease over the medium term as compared to the short term, long term, or both.

In some embodiments, the bioenergetics of a cell transplanted with mitochondria can increase or decrease over the long term as compared to the short term, medium term, or both.

In some embodiments, the increase in bioenergetics can range from about >1 fold to about 5 fold. In some embodiments, the increase in bioenergetics can range from about >1 fold to about 10 fold. In some embodiments, the increase in bioenergetics can range from about >1 fold to about 20 fold. In some embodiments, the increase in bioenergetics is about 2 fold.

In some embodiments, a decrease in bioenergetics can range from about 1.5-fold, 2-fold, 2.5-fold, 3-fold, 3.5-fold, 4-fold, a 4.5-fold, 5-fold, 5.5-fold, 6-fold, 6.5-fold, 7-fold, 7.5-fold, 8-fold, 8.5-fold, 9-fold, 9.5-fold, 10-fold, or a 20-fold. In some embodiments, the decreased in bioenergetics can range from >1 fold to about 5 fold. In some embodiments, the decrease in bioenergetics can range from about >1 fold to about 10 fold. In some embodiments, the decrease in bioenergetics can range from about >1 fold to about 20 fold. In some embodiments, the decrease in bioenergetics is about 2 fold.

In some embodiments, mitochondrial function can be affected by the number of passages (i.e., aging of the mitochondria) in a transplanted cell. An increased number of passages can result in a decrease in positive indicators of mitochondrial function, an increase in negative indicators of mitochondrial function, or both. Positive indicators of mitochondrial function include, without limitations, ATP production, and coupling efficiency. Negative indicators of mitochondrial function include, without limitations, proton leak. Thus, an increased number of passages can result in a decrease in positive indicators of mitochondrial function (e.g., ATP production, and coupling efficiency), an increase in negative indicators of mitochondrial function (e.g., proton leak), or both.

In some embodiments, it is anticipated improved bioenergetics with increased mitochondria up to a certain dosage is observed that eventually reaches a plateau or leads to reduced bioenergetics after a certain ratio of mitochondrial volume to cell volume. Thus, it is anticipated improved bioenergetics with increased mitochondria up to a certain dosage is observed that eventually reaches a plateau or leads to reduced bioenergetics due to constraints such as cell volume, cell expandability, etc. In other embodiments, it is anticipated that repeated treatment with varying dosage (depending on the extent of mitochondrial dysfunction) is needed to improve the bioenergetics.

In some embodiments, fabricated arrays of microposts can be used to measure contractile forces. In some embodiments, fabricated arrays of microposts are made via soft lithography by casting PDMS from silicon wafers with patterned SU-8 structures. In some embodiments, the dimensions are 2.3 μm in diameter, 7.2 μm in height, and 6 μm center-to-center spacing. In some embodiments, the dimensions can be varied by one having ordinary skill in the art according to need. In some embodiments, the diameter can range from about 0.46 μm to about 11.5 μm. In some embodiments, the height can range from about 1.44 μm to about 36 μm. In some embodiments, the center-to-center spacing can range from about 1.2 μm to about 30 μm.

In some embodiments, the contractile forces are measured by individual twitches recorded at the tips of the micropost. In some embodiments, the contractile forces are measured by individual twitches recorded at the tips of the micropost after 1 week. In some embodiments, the contractile forces are measured by individual twitches recorded at the tips of the micropost after about 7 days. In some embodiments, the contractile forces are measured by individual twitches recorded at the tips of the micropost between about 3.5 days and about 14 days.

In some embodiments the tip's position is compared to that of its base and the difference is multiplied by the bending stiffness to obtain a measure of contractile force. In some embodiments, the mean contractile force per cross-sectional area measured post-mitochondrial transplantation can be about 0.5, 1, 1.5, 2, 2.5, 3, 3.5, 4, 4.5, 5, 5.5, 6, 6.5, 7, 7.5, 8, 8.5, 9, 9.5, 10 or 20 mN/mm² per cell or any range defined by two of the preceding values.

In some embodiments, traction force microscopy can be used to measure contractile forces. In some embodiments, the contractile forces are measured by calculating the displacement of the embeded fluorescent beads used in traction force microscopy.

In some embodiments, fission and fusion dynamics of mt-DNA is measured using fluorescently-stabilized cell lines comprising fluorescently-labeled mitochondria. In some embodiments, fluorescently-labeled mitochondria are created through lipofectamine transfection of plasmid DNA constructs (or any expression vector known in the art, e.g. adenoviral expression vectors) that express one or more proteins targeted to the mitochondria fused to one or more fluorescent proteins. In some embodiments, the one or more proteins targeted to the mitochondria may be fused directly to the one or more fluorescent proteins. In some embodiments, the one or more proteins targeted to the mitochondria may be fused to the one or more fluorescent proteins via a linker. Non-limiting examples of fluorescent proteins include GFP, eGFP, mWasabi, superfolder GFP, TurboGFP, EBFP, EBFP2, ECFP, YFP, and the like. In some embodiments, high resolution or super resolution time-lapse microscopy can be used to ascertain at the dynamics of mitochondrial internalization.

Some embodiments are related to using mitochondrial transplantation to (1) replace dysfunctional mitochondria in diseased cells to improve cellular bioenergetics and function to mitigate mitochondrial dysfunction; (2) transplant mitochondria in normal cells to improve cellular bioenergetics and function post-mitochondrial transplantation with the intent of improving cellular performance. (3) to negatively modulate cellular bioenergetics to trigger the immune system to remove diseased cells such as cancerous cells

In some embodiments, the optimal cell seeding density for bioenergetic studies of mitochondrial transplantation can vary depending on the cell type to be transplanted, the source of mitochondria, or both. In some embodiments, the optimal cell seeding density ranges from about 4,000 cells to about 400,000 cells. In some embodiments, the optimal cell seeding density ranges from about 8,000 cells to about 200,000 cells. In some embodiments, the optimal cell seeding density is about 40,000 cells. In other embodiments, the optimal cell seeding density is about 50,000 cells.

Some embodiments are related to generating p° cells which are depleted of mitochondrial DNA as a mitochondrial disease model. In some embodiments, Seahorse analysis is used to determine whether mitochondrial transplantation in p° cells will induce a change in the cellular bioenergetics In other embodiments, cells or blood from patients with mitochondrial diseases, or other diseases with dysfunctional mitochondria, such as but not limited to diabetes, neurodegenerative diseases, and cancer will be used to determine whether mitochondrial transplantation will induce a change in the cellular bioenergetics.

In some embodiments, testing whether cells with abnormal mitochondria will adopt the normal mt-DNA from the transplanted mitochondria is disclosed.

In some embodiments, mitochondria are transplanted into mitochondrial depleted cells.

In some embodiments, mitochondria isolated from one species are transplanted into cells of another species.

In some embodiments, mitochondria are transplanted into an immune cell, e.g., T-cell, a B-cell, a monocyte and a natural killer (NK) cell.

In some embodiments, mitochondria are transplanted into immortalized T cells, e.g., Jurkat Cells.

### EXAMPLE 1

### Bioenergetics Consequences of Mitochondrial Transplantation in Cardiomyocytes

We quantitated the bioenergetics consequences of mitochondrial transplantation into cardiomyocytes up to 28 days using a seahorse flux analyzer. Compared to the control, we observed a statistically significant improvement in basal respiration and ATP production 2-day post-transplantation, accompanied by an increase in maximal respiration and spare respiratory capacity, although not statistically significantly. However, these initial improvements were short-lived and the bioenergetic advantages return to the baseline level in subsequent time points.

This study for the first time shows that transplantation of non-autologous mitochondria from healthy skeletal muscle cells into normal cardiomyocytes leads to short-term improvement of bioenergetics indicating “super-charged” state. However, over time these improved effects disappear, which suggests transplantation of mitochondria may have a potential application in settings where there is an acute stress. We anticipate the improved bioenergetics is short-lived because the mitochondria were transplanted into normal cells, which does not have a need for the increased mitochondrial content, and the retainment of enhanced bioenergetics could be longer in cells with mitochondrial dysfunction.

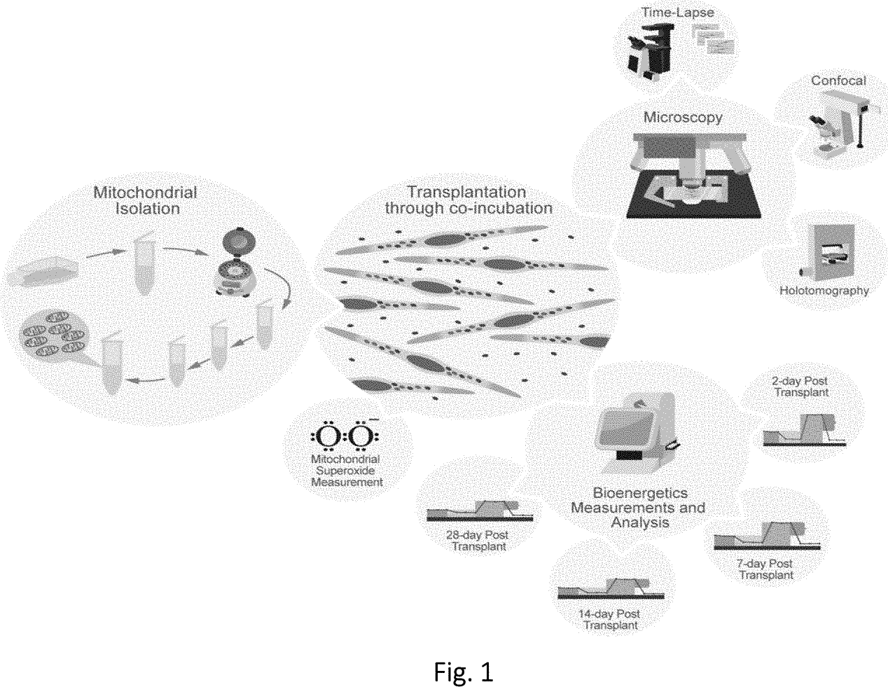

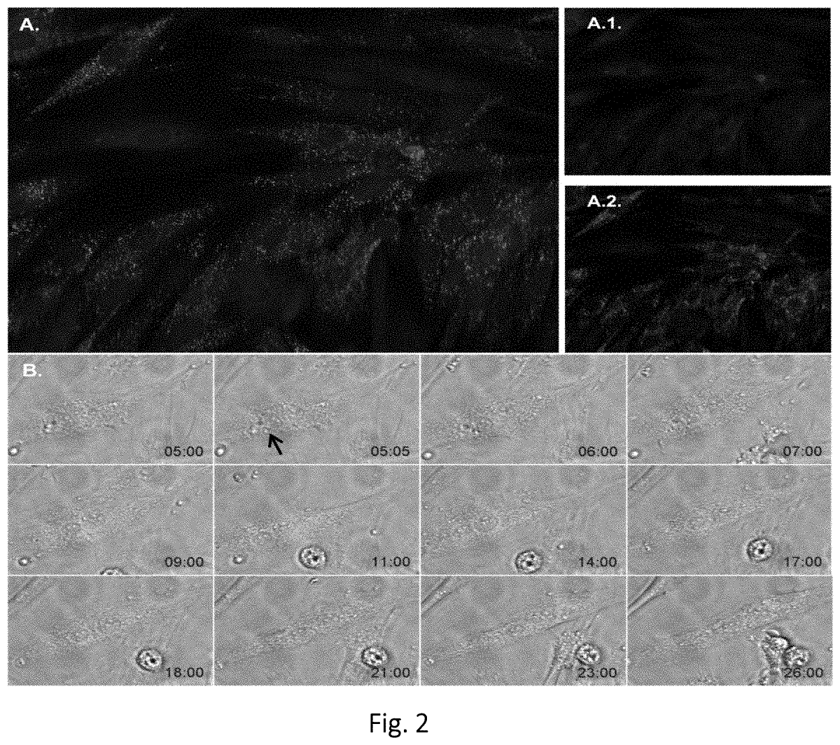

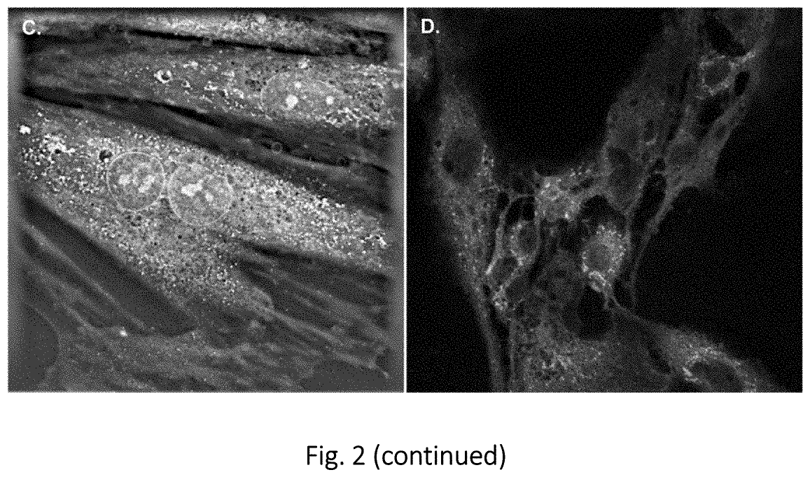

In our studies, as schematically shown in FIG. 1, we isolated mitochondria from H9c2 for autologous transplantation (FIG. 2, A and B), L6 cells for non-autologous (C) and interspecies transplantation (D) using a commercially available isolation kit for cultured cells (ThermoFisher Scientific, Waltham, Mass.). Cardiomyocytes were plated at 50K on a μ-slide 8-well ibiTreat slide (ibidi, Martinsried, Germany) the day before mitochondrial isolation for imaging purposes, 40K cells onto a Seahorse microchamber plate (Agilent Technologies, Santa Clara, Calif.) the day before running the Seahorse assay, and 40K cells onto a 96-well plate the day before running the mitoSOX superoxide production assay. For the holotomographic microscopy (FIG. 2, C) a 35 mm dish (ibidi, Martinsried, Germany) was used.

Mitochondrial labeling and imaging. Native mitochondria were labeled with MitoTracker Green FM with ex/em 490/516 nm at 37° C. for 45 minutes and washed twice with sterile PBS (ThermoFisher Scientific, Waltham, Mass.). The isolated mitochondria were labeled with pHrodo Red SE with ex/em 560/585 nm for 30 minutes at 4° C., washed twice with sterile PBS (ThermoFisher Scientific, Waltham, Mass.), and then co-incubated with H9c2 cells. The nucleus was labeled with NucBlue Live with ex/em 360/460 nm (ThermoFisher Scientific, Waltham, Mass.). A time-lapse microscopy was subsequently conducted with both fluorescence and phase contrast/DIC channels to capture the dynamic behavior of mitochondrial internalization (FIG. 2, B). Images were acquired either using a widefield (Olympus IX83) or Confocal microscope (Zeiss LSM 780). The respective data were analyzed using ImageJ and IMARIS software.

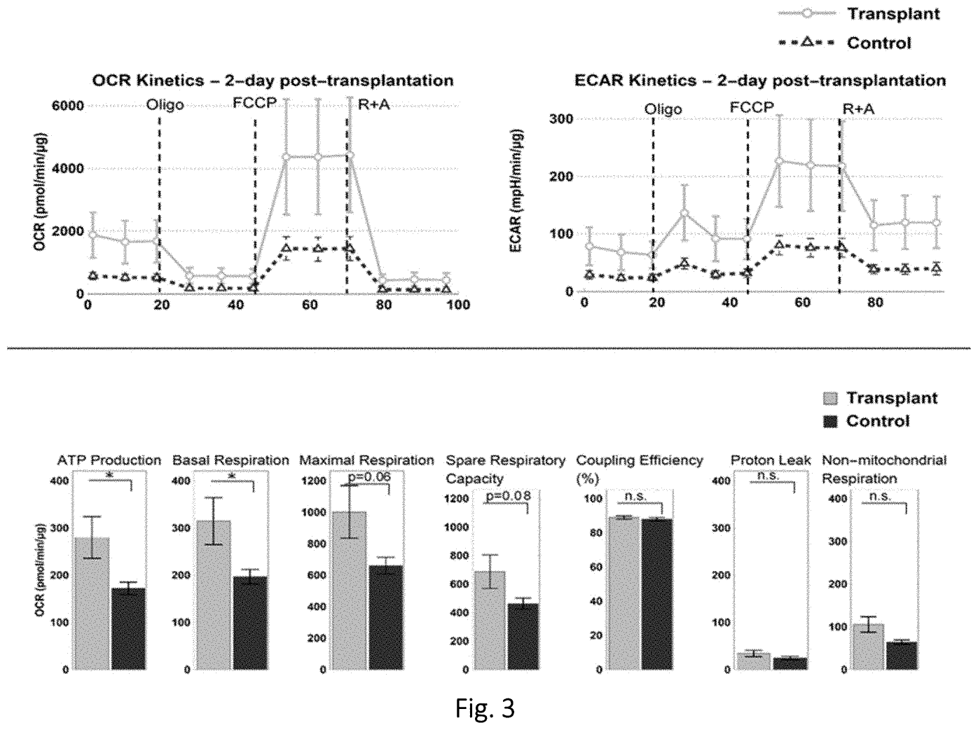

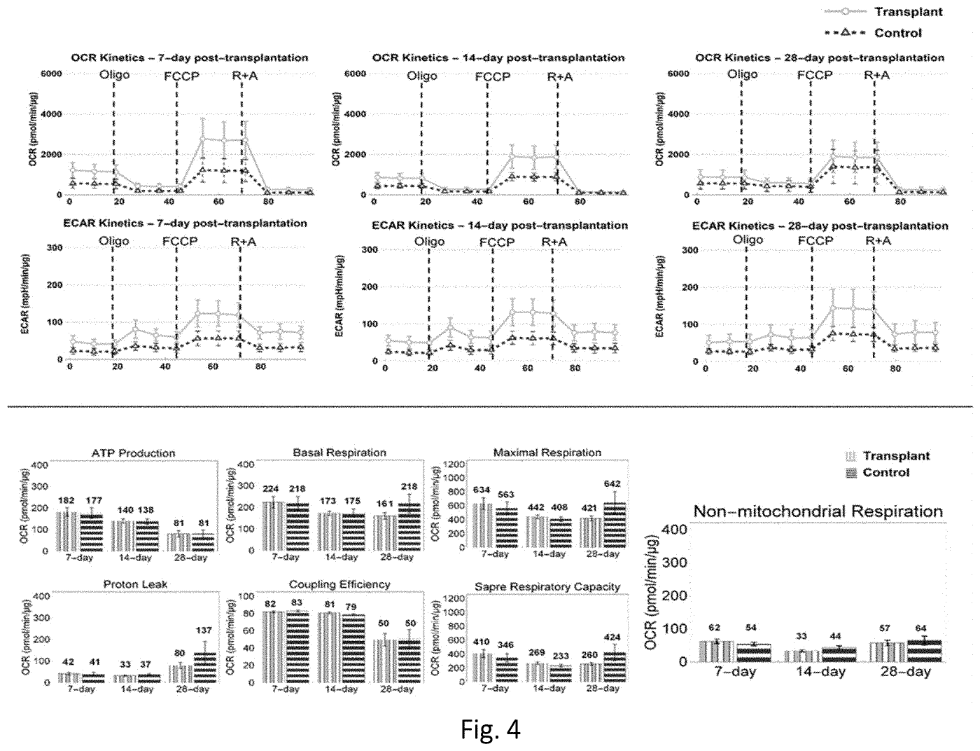

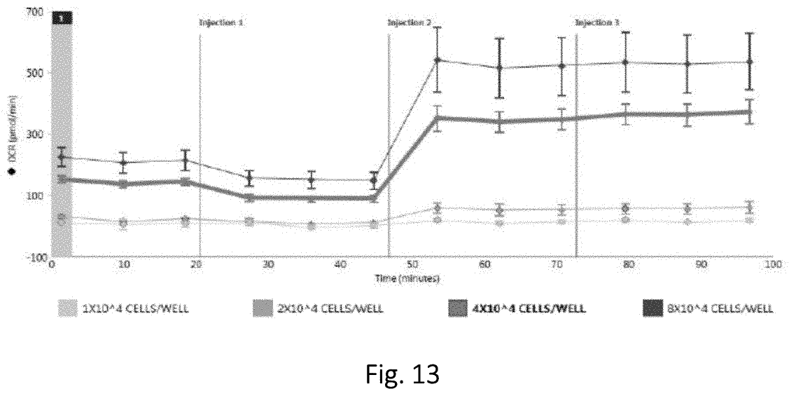

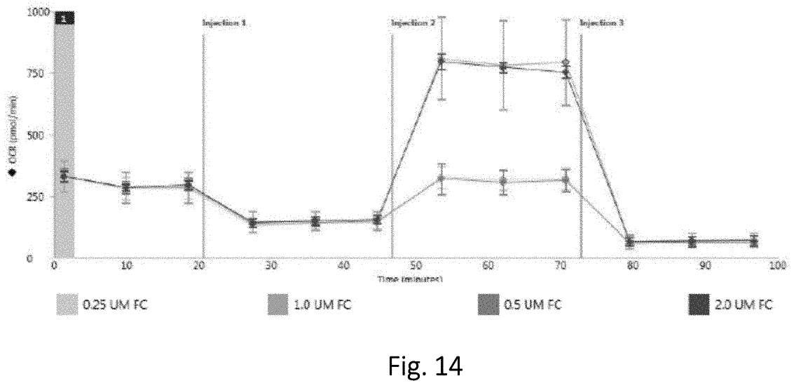

To investigate the bioenergetics consequences of mitochondrial transplantation, oxygen consumption rate (OCR) and extracellular acidification (ECAR) rates were measured post transplantation using a Seahorse XF24 Flux Analyzer. First, H9c2 cells were plated at seeding densities of 10K, 20K, 40K, and 80K to find the optimized cell seeding density which was experimentally determined to be 40K (FIG. 13). This was followed by drug optimization tests, in which the optimal final well concentrations were experimentally determined to be 1 μM for Oligomycin (Oligo), 1 μM for FCCP (FIGS. 14) and 0.5 μM of Rotenone +0.5 μM of Antimycin (R+A). Mitochondria from L6 skeletal cells (p.9) were isolated (from 100 cells per one recipient cell) using a commercially available isolation kit and transplanted into H9c2 cardiomyocyte cells (p.10). Isolated mitochondria were co-incubated with H9c2 cells for 24 hours. After that any mitochondria not internalized into cardiomyocytes were removed by changing the culture media. The Seahorse assays were performed in a Seahorse XF DMEM medium supplemented with glucose to a final concentration of 10 mM, sodium pyruvate to 1 mM, and L-glutamine to 2 mM. Bioenergetics measurements were performed, after sequential treatment with Oligomycin, FCCP, and Rotenone/Antimycin A (Agilent Technologies, Santa Clara, Calif.), 2-day (FIG. 3), 7-day (FIG. 4), 14-day (FIGS. 4), and 28-day (FIG. 4) post-transplantation and compared to that of the control group to investigate the effect of mitochondrial transplantation in two sets of independent studies, with a total of 28 observation for the experimental group and 12 for the control group. Raw data was normalized to total protein using Micro BCA protein Assay (ThermoFisher Scientific, Waltham, Mass.).

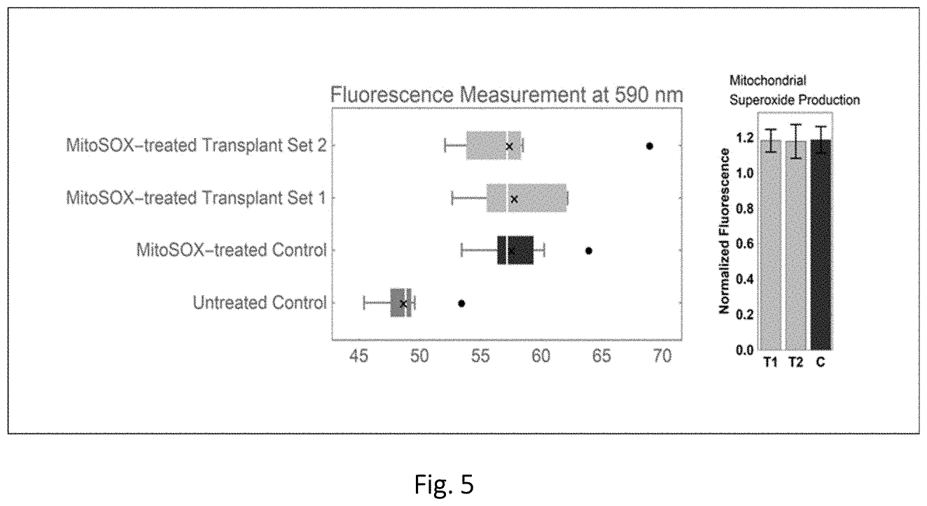

Quantification of mitochondrial Superoxide Production. To assess the production of superoxide by mitochondria, MitoSOX Red Mitochondrial Superoxide Indicator (ThermoFisher Scientific, Waltham, MA) was used. At 2-day post-transplantation the cells were incubated with MitoSOX Red (5 μM) at 37° C. for 10 minutes. The cells were then washed three times with sterile PBS. Using a fluorescence plate reader, MitoSOX Red was excited at 485 nm and fluorescence emission was measured at 590 nm. Fluorescence values were recorded for two groups of independent transplantation experiments and compared to a control group, with 10 observations for each group (FIG. 5).

Statistical Analyses: An unpaired two-tailed unequal variance t-test (Welch's Test) was used for the mean values of Seahorse bioenergetics data in Microsoft Excel. A Welch's Test, which is a more stringent test than the student t-test was used. This was done to account for the possibility of mitochondrial transplantation changing the bioenergetics both positively and negatively (two-tailed), possible different distributions of the control and transplant groups (unequal variance), and that the measurements were taken independently (unpaired). To check for normality, qq plots of the data were generated in R. The distribution in transplant appears in fact to be different from the control group. Most observations fell within the 95% confidence interval, and overall the data were reasonably normally distributed. Results with p<0.05 were considered as statistically significant. No statistical methods were used to predetermine the sample size. Sample size was based on experimental feasibility for proof of concept. The graphs presented in FIGS. 3 to 6 were generated in Mathematica (Wolfram Research, Champaign, Ill.). Data from bar graphs presented in FIGS. 3, 4, and 6 are means±SEM and data from kinetic profiles in FIGS. 3 and 4 are mean±SD from n=2 independent experiments each with 14 biological replicates for the experimental transplant group and 6 biological replicates for the control group. For the 28-day post-transplantation experiment, 27 observations were recorded versus the 28 for all the other time-points, due to measurement failure in one of the wells.

A Welch's test was used to compare post-transplant cardiomyocytes' mean superoxide production to that of the control at the 5% level. Fluorescence values after MitoSOX-treatment were recorded from n=2 independent transplant groups and compared to n=1 control group, each with 10 biological replicates. No statistical methods were used to predetermine sample size. The fluorescence values are displayed as Box and Whisker plots in FIG. 5. The fluorescence measurement from the mitoSOX-treated control and transplant groups were normalized to the mean fluorescence measurement value of the untreated control. Mitochondrial superoxide production was reported as the normalized fluorescence values ±SD and presented as bar graphs in FIG. 5.

### RESULTS

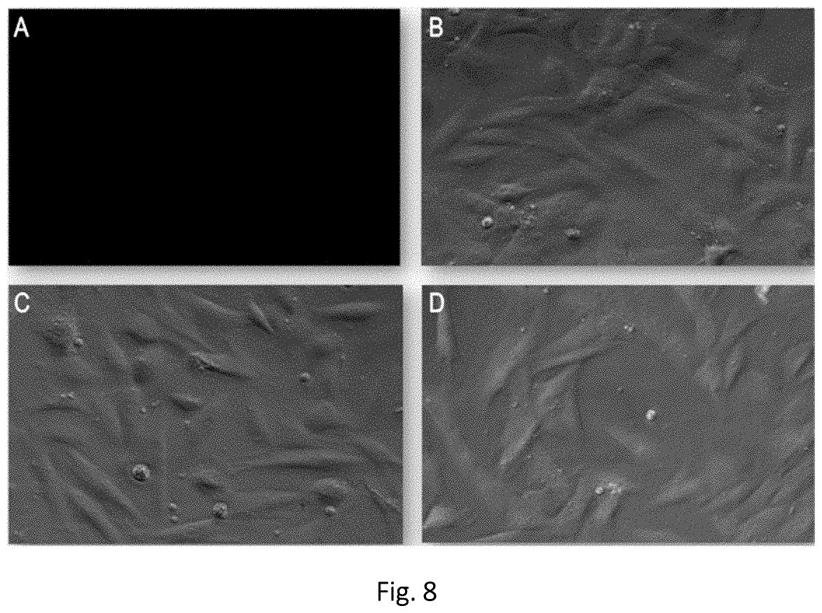

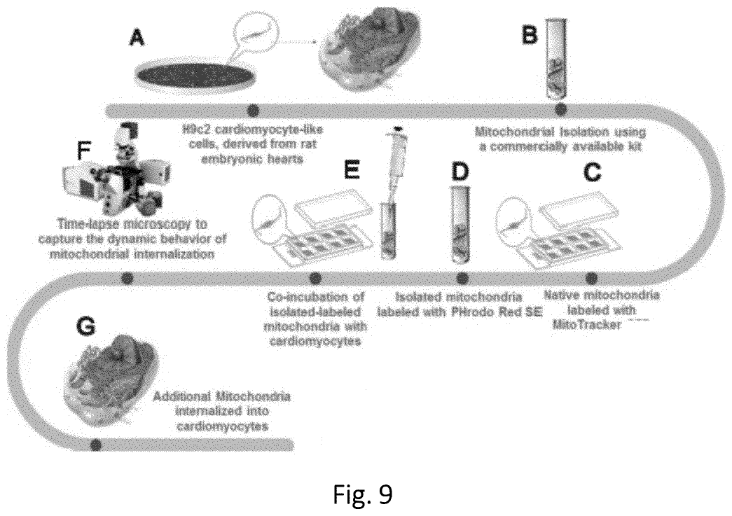

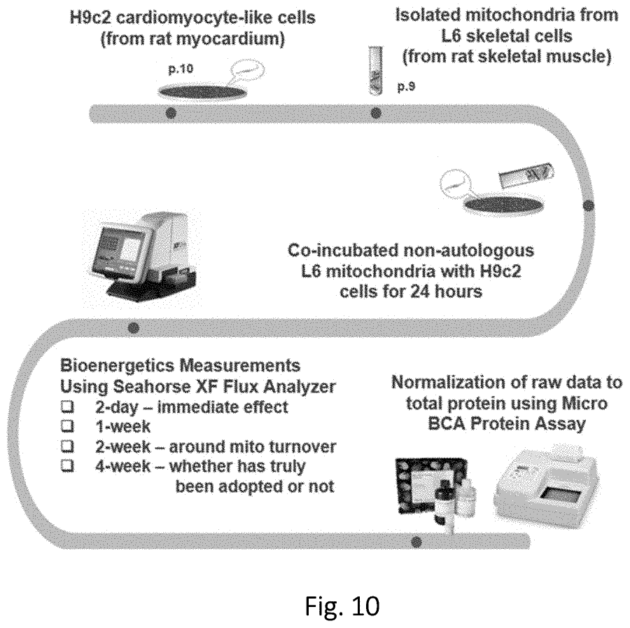

Inspired by the endosymbiosis theory of mitochondrial origin, we hypothesized and tested whether co-incubation of isolated mitochondria with cells would allow for mitochondria' s uptake by the cell and enhanced cellular bioenergetics state. First, we evaluated autologous mitochondrial transplantation of rat cardiomyocyte H9c2 cells (ATCC, Manassas, Va.). Based on a 28-hour time-lapse study (FIG. 2, A and B), we observed mitochondrial internalization at various time points and visualized the dynamics of mitochondrial internalization in time. FIG. 2, B shows representative images demonstrating the cellular uptake of pHrodo Red Succinimidyl Ester (SE) labeled mitochondria (black arrow). The cells remained viable for the duration of the experiment while the internalized mitochondria appeared to undergo fission and fusion dynamics based on the propagation of the pHrodo Red signal to the entire cell (FIG. 2, B). From a mechanistic perspective, in labeling the isolated mitochondria with pHrodo Red SE, the SE group interacts with the amine group on the mitochondria and forms a covalent amide bond. Nevertheless, to ensure the specificity of the detected signal representing mitochondrial internalization, we performed a set of control experiments with four groups as described in Table 1. All four groups lacked isolated mitochondria. Collectively, our experiments showed that the pHrodo dye by itself cannot passively internalize, and consequently the pHrodo-labeled autologous mitochondria actively internalize into the cells (FIG. 8).

TABLE 1
Mitochondrial Transplantation Validation Experiment.

| | Group Descriptions |
|---|---|
| 1 | pHrodo Red SE dye present in the media, with no cell plated |
| 2 | cells in the absence of pHrodo Red SE dye in the media. |
| 3 | cells in the presence of pHrodo Red SE dye in the media. |
| 4 | cells in the presence of pHrodo Red SE dye in the media, |
| | and native mitochondria fluorescently labeled with |
| | MitoTracker Green FM to control for proper function of |
| | microscope and fluorescent signal detection. |

Next, we tested whether cells would similarly uptake non-autologous mitochondria (FIG. 2, C). Mitochondria isolated from rat L6 skeletal muscle cells (ATCC, Manassas, Va.) were internalized by the rat cardiomyocyte H9c2 cells. This set of experiments shows that non-autologous mitochondrial transplantation between the two rat cell lines is possible (FIG. 2. C).

Lastly, we investigated interspecies mitochondrial transplantation by using isolated mitochondria from the rat L6 cells and transplanting them into the human ARPE-19 retinal cells (ATCC, Manassas, Va.) (FIG. 2, D). Similar to the previous experiments, mitochondria were successfully internalized into the cells and the feasibility of mitochondrial transplantation from rat to human cells was demonstrated.

We then hypothesized and tested whether the internalization of the transplanted non-autologous mitochondria leads to enhanced bioenergetics, by conducting Cell Mito Stress Test using a Seahorse XF24 Flux Analyzer. We measured the oxygen consumption rate (OCR), which represent the rates of oxidative phosphorylation and extracellular acidification rate (ECAR), which denotes the summation of acid (H+) produced from glycolysis and the TCA cycle (via CO2), when obtained through Mito Stress Test. Subsequently, we compared the results to the control groups with no transplantation event. Baseline and post-transplant rates were measured at four different time points: post 2-day to measure acute effects (FIG. 3), post 7-day, post 14-day to check bioenergetic indices around mitochondria turnover, and finally post 28-day to check for long-term effect (FIG. 4). We observed an upward shift in OCR kinetics 2-day post-mitochondrial transplantation compared to the baseline (FIGS. 3). Compared to the control, we observed a statistically significant improvement in basal respiration and ATP production 2-day post-transplantation (p=0.031 and p=0.025, respectively; FIG. 3). Maximal respiration and spare respiratory capacity—the cell's bioenergetics reserve in meeting a situation of high demand or acute/chronic stress—were also enhanced post 2-day, although not statistically-significantly, p=0.060 for maximal respiration and p=0.080 for spare respiratory capacity (FIG. 3). Coupling efficiency was unchanged as both transplanted and native mitochondria were healthy (FIG. 3).

Mitochondrial superoxide production was measured 2-day post transplantation using MitoSOX Red in control and transplant groups, and no significant difference was observed among the groups (FIG. 5).

### Discussion

H9c2 cells are phenotypically purely aerobic as the heart tissue has a high-energy demand, which is more efficiently met by aerobic respiration and oxidative phosphorylation. Increase in OCR kinetics 2-day post-transplantation is indicative of improved bioenergetics, which is justified by more oxidative phosphorylation due to increased mitochondrial content (FIG. 3). Based on our bioenergetics results, it appears that in the normal cells, mitochondrial transplantation leads to acutely enhanced bioenergetics. However, these initial effects seem to diminish over time and return to that of the baseline control (FIG. 4).

In addition to OCR, we evaluated the ECAR from the Mito Stress Test (FIG. 3 and FIG. 4). In many cell types, mitochondrial activity (TCA cycle) that leads to CO2 production is a significant source of extracellular acidification (Mookerjee S A, Goncalves R L S, Gerencser A A, Nicholls D G, Brand M D. The contributions of respiration and glycolysis to extracellular acid production. Biochimica et Biophysica Acta (BBA)-Bioenergetics. 2015; 1847:171-181). Since in our observation the ECAR significantly decreased after R+A injection, we suppose that in our samples the acidification was generated through the TCA cycle. However, from Mito Stress Test, only qualitative interpretation of the changes in ECAR data can be made for the kinetics after the injection of rotenone and antimycin. If the ECAR significantly decreases (i.e., low residual ECAR) in response to R+A, we infer the ECAR (prior to the injection of R+A) is primarily being generated via CO₂ production from the TCA cycle. If the ECAR remains constant and/or increases in response to R+A, then the ECAR prior to R+A treatment is primarily being generated via glycolysis (Divakaruni A S, Paradyse A, Ferrick D A, Murphy A N, Jastroch M. Chapter sixteen—analysis and interpretation of microplate-based oxygen consumption and pH data. In: Murphy A N, Chan D C, eds. Methods in enzymology. Academic Press; 2014:309-354). The exogenous pyruvate and glutamine supplemented in the media can be directly shunted into the TCA cycle, which lead to CO₂ production independent of the CO₂ produced from the pyruvate generated from glucose through glycolysis. Since we observed a decrease in response to R+A in both control and transplant groups, we infer that that CO₂ is primarily being generated via TCA cycle in this cell type. Another viewpoint indicates that in both control and transplant groups, the cells were burning through the exogenous pyruvate and glutamate. However, on the contrary, we interpret our ECAR data (FIGS. 3 and 4), such that the increase in ECAR kinetics in transplant vs. the control group (FIG. 3) is due to CO₂ production from the increased TCA cycle in transplant group. Glycolytic Rate Assay can delineate the sole contribution of glycolysis or CO2 to ECAR, which we did not perform in the current experiments.

Previously, Finck and Kelly (Finck B N, Kelly D P. Peroxisome proliferator-activated receptor gamma coactivator-1 (pgc-1) regulatory cascade in cardiac physiology and disease. Circulation. 2007; 115:2540-2548) reported that the energetic needs of the cell finely tune the mitochondrial abundance, and too many or too few mitochondria may lead to pathology. In their review of myocardial energy metabolism, they associated myocardial diseases with perturbations of mitochondrial biogenesis. PGC-1α is a protein that drives mitochondrial biogenesis in cardiomyocytes by activating the nuclear respiratory factor 1, which in turn triggers the expression of activated mitochondria, leading to transcription and replication of the mitochondrial genome. Mice lacking PGC-1α present with signs of heart failure. Alternatively, downregulation of PGC-1α in mice leads to hypertrophic cardiomyopathy, whereas prolonged overexpression of PGC-1α in mice leads to increased biogenesis activity and mitochondrial ultrastructural abnormalities, which ultimately leads to death from heart failure (Finck B N, Kelly D P. Peroxisome proliferator-activated receptor gamma coactivator-1 (pgc-1) regulatory cascade in cardiac physiology and disease. Circulation. 2007; 115:2540-2548). Based on our observations, the enhanced bioenergetics state is temporary, which may imply that either the cells' mitochondrial content is returned to physiological levels, or the overall mitochondrial activities are reduced to reflect the cell's bioenergetics needs. Since the spare respiratory capacity, which measures the cell's capacity in meeting a situation of high energy demand, is not significantly changed in the transplant vs. the control, it is less likely that the overall mitochondrial activities have dampened and more likely that mitochondrial content has returned to physiological levels. Our observation corroborates with the findings of Finck and Kelly, and accordingly, we hypothesize that a normal cell clears any excess mitochondria keeping the mitochondrial content at physiological levels, as the plausible mechanism for the return of the bioenergetics indices to the baseline levels in long-term. In case of an adjustment of the mitochondrial content, it is yet unclear whether the newly transplanted mitochondria is cleared from the cell or if the transplanted mitochondria have been adopted by the host and the total mitochondrial level regardless of the recipient or donor status is reduced to bring the bioenergetics levels back to physiological state. This question can be addressed through post-transplantation, time-course mitochondrial DNA sequencing.

Moreover, the long-term bioenergetics profile suggests that there is no negative bioenergetic consequences due to mitochondrial transplantation and that the intervention can be considered safe for the cell. The question remained to be answered is whether cells with damaged mitochondria would take advantage of the newly introduced mitochondria or not, as those cells could uptake and adopt the mitochondria differently than a normal cell, which is already able to efficiently meet its bioenergetics demands.

Non-mitochondrial respiration—an index that measures oxygen consumed by the cell via processes that are non-mitochondrial—significantly increased post 2-day (p=0.033) and returned to the baseline post 7-day, 14-day and 28-day (p >0.05 for all; FIG. 3 and FIG. 4). Non-mitochondrial respiration is not well-understood or well-studied (Hill B G, Benavides G A, Lancaster J R, Jr., Ballinger S, Dell'Italia L, Jianhua Z, Darley-Usmar V M. Integration of cellular bioenergetics with mitochondrial quality control and autophagy. Biol Chem. 2012; 393:1485-1512) Some non-mitochondrial processes that use oxygen are superoxide production (with deleterious effects for mitochondria (Boveris A, Chance B. The mitochondrial generation of hydrogen peroxide. General properties and effect of hyperbaric oxygen. Biochem J. 1973; 134:707-716; and Turrens J F. Mitochondrial formation of reactive oxygen species. J Physiol. 2003; 552:335-344) and hydrogen peroxide production (Boveris A, Oshino N, Chance B. The cellular production of hydrogen peroxide. Biochem J. 1972; 128:617-630). It is known that the hydrogen peroxide protects the cell from superoxide production given that it is properly degraded by catalase; otherwise it can break apart and form hydroxyl radicals. The increase in non-mitochondrial respiration could either suggest an improved overall metabolism or stress.

In a normal tightly coupled electron transport chain, approximately 1-3% of the consumed oxygen is incompletely reduced, which can lead to superoxide production as a result of the interaction between the leaky electrons and molecular oxygen (Jastroch M, Divakaruni A S, Mookerjee S, Treberg J R, Brand M D. Mitochondrial proton and electron leaks. Essays Biochem. 2010; 47:53-67; and Batandier C, Fontaine E, Kériel C, Leverve X M. Determination of mitochondrial reactive oxygen species: Methodological aspects. Journal of Cellular and Molecular Medicine. 2002; 6:175-187). Since we had observed a statistically-significant enhancement in bioenergetics due to increased oxidative phosphorylation at 2-day post-transplantation, we used the mitochondrially-targeted derivative of hydroethidine (HE)—MitoSOX Red—to detect and quantitate the level of superoxide produced by mitochondria in post-transplant and control groups 2-day after transplantation. Compared to control, no statistically-significant difference was found in superoxide production in the post-transplant group (p=0.99 and p=0.83 for set 1 and 2 transplantations, respectively; FIG. 5). The reported superoxide production was not assessed for the same Seahorse experiment, but a representative one.

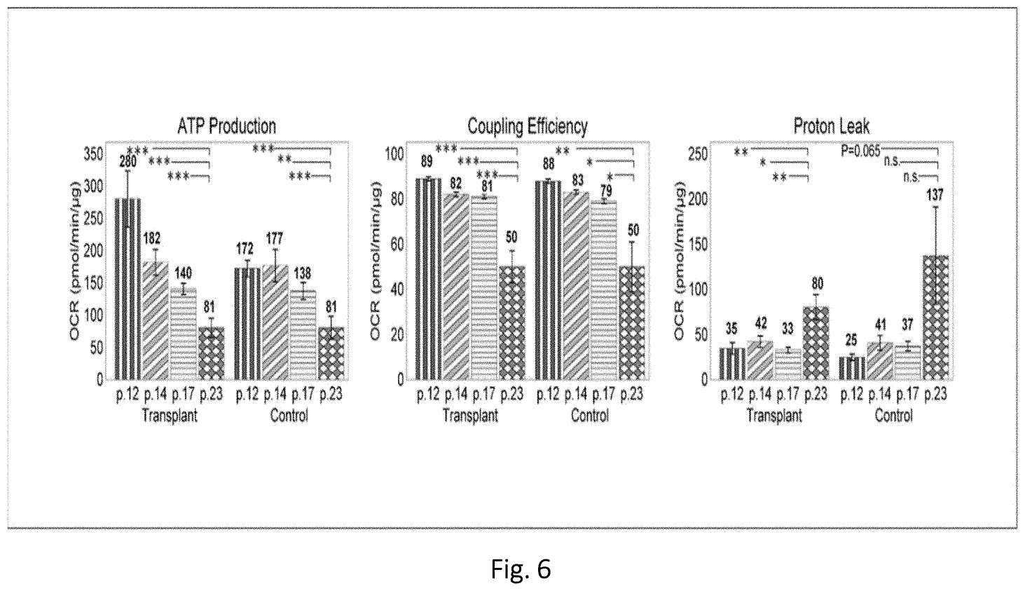

Finally, we observed a reduction in mitochondrial function in the studied groups with increasing the passage number. ATP production and coupling efficiency, positive indicators of bioenergetics, significantly decreased with increasing passage number, while proton leak, a negative indicator of bioenergetics, significantly increased (FIG. 6). This observation corroborates with the findings of a previous study by Witek et al. (Witek P, Korga A, Burdan F, Ostrowska M, Nosowska B, Iwan M, Dudka J. The effect of a number of h9c2 rat cardiomyocytes passage on repeatability of cytotoxicity study results. Cytotechnology. 2016; 68:2407-2415), where they observed an increase in oxidative stress in cells with passaging of cardiomyocytes. It appears that mitochondrial function is reduced in cells with higher passage number and thus these cells are not representative of the initial passages. H9c2 cell-line is a well-established cell line used for assessing drug-induced cardiotoxicity (Witek P, Korga A, Burdan F, Ostrowska M, Nosowska B, Iwan M, Dudka J. The effect of a number of h9c2 rat cardiomyocytes passage on repeatability of cytotoxicity study results. Cytotechnology. 2016; 68:2407-2415) and this observation indicates that only lower passages of this line should be used to consider mitochondrial adverse events.

Since the focus of the presented study has been on mitochondrial oxidative phosphorylation, the Mito Stress Test was conducted. Thus, the ECAR kinetics presented in FIGS. 3 and 4 represent the summation of acid (H+) produced from glycolysis and CO2 produced from the TCA cycle. Accordingly, the ECAR data in this study is not a direct measurement of glycolysis. The XF Glycolytic Rate Assay may be performed to quantitate the changes due to glycolysis.

### CONCLUSION

Our studies show that normal cellular bioenergetics is acutely enhanced after mitochondrial transplantation. The results delineate the potential of mitochondrial transplantation for clinical application in settings where there is an acute stress that would benefit from a boost in cellular bioenergetics. These results provide a basis for using mitochondrial transplantation to modulate bioenergetics profiles in cells with mitochondrial dysfunction.

### EXAMPLE 2

### Mitochondrial Transplantation Protocol

1. Cell Culture

A. Materials:
Desired mitochondria-donor cells and recipient cells Dublecco's Modified Eagle's Medium (DMEM) (ATCC, Manassas, Va.)
Fetal Bovine Serum (FBS) (ATCC, Manassas, Va. )
Penicillin-Streptomycin Solution (pen-strep) (ThermoFisher Scientific, Waltham, Mass.)
Trypsin-EDTA (0.25%), phenol red (ThermoFisher Scientific, Waltham, Mass.)
Nunc Lab Tek II CC2 Chamber Slide System (ThermoFisher Scientific, Waltham, Mass.)
T75 Corning Cell Culture Flasks, vented cap
Dimethyl Sulfoxide (DMSO)
P-2, P-10, P-20, P-200, and P-1000 micropipetters and pipette tips
5.0 mL, 10.0 mL, 20.0 mL Disposable pipettes and pipette controller
Humid CO₂ incubator (37° C., 5% CO₂)
Centrifuge
Microscope
Countess Automated Cell Counter and Cell Countess Slides
Trypan Blue
70% Ethanol
Mr. Frosty Freezing Container
cryovials

B. Protocol:

### Day 0

1) Upon receipt of the vial initiate the culture as soon as possible to ensure the highest level of viability. If not, store in liquid nitrogen until ready to use.
2) Place the complete growth media in the 37° C. water bath to warm the solution. Complete growth media consists of DMEM, FBS to a final concentration of 10% and pen-strep to a final concentration of 1%.
3) Turn on the cell culture hood blower (and light if separate switches), and disinfect all the surfaces by spraying them with 70% ethanol and wiping clean. Disinfect each item (such as pipettes, pipette tips, etc.) placed in the cell culture hood by also spraying them with 70% ethanol and wiping clean. Keep the workspace in the cell culture hood clean and uncluttered, and keep everything in direct line of sight. Designate a clean side and a waste side for yourself. For a right-handed person the wide, clear space in the center is the work space, the tube rack should be in the rear middle, the reagents and media should be kept on the rear right, the pipettes in the front right, and all waste on the rear left.
4) Spray a T75 vented-cap corning cell culture flask with 70% ethanol and transfer 12.0 mL of complete growth medium to it. Place the flask in a humid CO₂ incubator (37° C., 5% CO₂). This is to ensure that the media has been warmed up and the normal pH has reached at least 10 minutes before any cells are added to the flask, so that the cells will not be stressed.
5) To initiate the culture, start by placing the vial in an O-ring, keeping the cap out of the 37° C. water bath and quickly (1-2 minutes) thaw the cells by gently swirling the vial in the water.
6) After the contents are thawed, spray the vial with 70% ethanol and carry out the remaining steps in the cell culture hood.
7) Transfer the vial contents to a centrifuge tube containing 9.0 mL of pre-warmed complete growth medium, and spin at 125 rcf for 5 minutes.

8) Aspirate the supernatant and re-suspend the cell pellet in 1.0 mL of pre-warmed complete growth medium.
9) Using a p-1000 pipette, slowly dispense (drop by drop) the cells in the cell culture flask so that the cells are uniformly distributed on the flask. Check the cells under the microscope.
10) Incubate the flask in a humid CO₂ incubator (37° C., 5% CO₂), and change the media the following day.

### Day 1

1) Place the complete growth media in the 37° C. water bath to warm the solution.
2) Turn on the cell culture hood blower (and light if separate switches), and disinfect all the surfaces by spraying them with 70% ethanol and wiping clean. Disinfect each item (such as pipettes, pipette tips, etc.) placed in the cell culture hood by also spraying them with 70% ethanol and wiping clean. Keep the work space in the cell culture hood clean and uncluttered, and keep everything in direct line of sight. Designate a clean side and a waste side for yourself. For a right-handed person the wide, clear space in the center is the work space, the tube rack should be in the rear middle, the reagents and media should be kept on the rear right, the pipettes in the front right, and all waste on the rear left.
3) After ˜15 minutes, when the media has warmed, spray the container with 70% ethanol and place it in the cell culture hood.
4) Take out the cell culture flask from the incubator, check the cells under the microscope for attachment and distribution, spray it with 70% ethanol, and place it in the cell culture hood.
   a. Tilt the flask to its side and aspirate the media from a corner (using pipettor and disposable pipette).
5) Using a new disposable pipette, slowly dispense 12.0 mL of fresh pre-warmed complete growth medium into the cell culture flask. Place the flask back into the humid CO₂ incubator (37° C., 5% CO₂).
6) Monitor the cells' confluency over the next few days, and subculture when the cells are ˜70-80% confluent (H9c2 cell line will lose its myoblastic properties if it reaches confluency), which usually happens on Day 3.

### Day 3

1) Place the complete growth media and trypsin solution in the 37° C. water bath.
2) Turn on the cell culture hood blower (and light if separate switches), and disinfect all the surfaces by spraying them with 70% ethanol and wiping clean. Disinfect each item (such as pipettes, pipette tips, etc.) placed in the cell culture hood by also spraying them with 70% ethanol and wiping clean. Keep the work space in the cell culture hood clean and uncluttered, and keep everything in direct line of sight. Designate a clean side and a waste side for yourself. For a right-handed person the wide, clear space in the center is the work space, the tube rack should be in the rear middle, the reagents and media should be kept on the rear right, the pipettes in the front right, and all waste on the rear left.
3) For each flask of cells that you plan to subculture, use the appropriate subculture ratio to decide on the number of flasks required, and transfer the appropriate amount of complete growth medium to it. Place the flask in a humid CO₂ incubator (37° C., 5% CO₂). For example, the subculture ratio for H9c2 cardiomyocyte-like cells is 1:2. Alternatively, you can wait to determine the number of flasks necessary, by counting the cells using the Countess Automated Cell Counter or by using a Hemocytometer. The seeding density for this cell line is 1×10⁴ cells/cm².
4) After ˜15 minutes, when the solutions have warmed, spray the containers with 70% ethanol and place them in the cell culture hood.
5) Under the microscope, check the T75 flasks for appropriate confluency. For example, H9c2 should not be allowed to reach 100% confluency or it will lose its myoblastic properties. Passage when ˜70-80% confluent, which is usually achieved on day 3.
6) Aspirate media.
7) Add 3.0 mL of warmed trypsin to the T75 flask and place it in the incubator for 2 minutes.
8) Remove T75 flask from incubator and check under the microscope for detached cells.
9) If cells are not rounded and free floating in suspension (less than 90% detached), gently tap the flask (to expedite cell detachment) and/or place back in the incubator for additional 1-2 minutes. Use microscope to check for cell detachment.
10) Add 6.0 mL of warmed complete growth media to neutralize the trypsin solution.
11) Transfer the cell suspension from the T75 flask into a conical (depending on how many flask you are subculturing at the same time choose a 15.0 mL or 50.0 mL conical).
12) Centrifuge conical at 125 rcf for 5 minutes.
13) Aspirate supernatant leaving cell pellet untouched.
14) Resuspend cell pellet in 1.0 mL media. Carefully break up cell pellet by gently pipetting using the p-1000 micropippette.
15) To count the cells, thoroughly mix cell suspension and transfer 10 μL into a 0.6 mL microcentrifuge tube. Take the microcentrifuge tube with cell suspension to cell countess and add 10 μL Trypan Blue. Mix well, and transfer 10 μL of cell/Trypan Blue to one side of cell countess slide and count cells.
   Total number of cells=alive cells/mL*resuspension volume
   Total number of cells needed=seeding density*TC area
   Required cell suspension volume=number of cells needed/cell density
16) If continuing to subculture and seed use 2.0 mL for each T75 flask to resuspend the pellet since you will split it 1:2 (for H9c2), so that you will use 1.0 mL for each new T75 flask. If freezing the cells, use 1.0 mL of cryoprotectant solution, 5% DMSO (950 μL of complete growth medium and 50 μL of DMSO) instead for each vial you wish to freeze the cells and store in liquid nitrogen.
17) Take out the T75 flasks out of the incubator, spray with 70% ethanol and place in the cell culture hood.
18) Mix the cell suspension and distribute the cells evenly drop by drop using a p-1000 micropipetter.
19) Check under the microscope for uniform cell distribution. 20) If freezing the cells, place label the cryovial(s) and place in a labeled Mr. Frosty.
21) Place Mr. Frosty in −80° C. freezer for a minimum of 6 hours and no more than 48 hours before moving it to Liquid Nitrogen Storage Tank.
22) When placing the cryopreserved cells in the liquid nitrogen tank, wear safety glasses, a face shield, and protective gloves in addition to PPEs above.
23) Fill out the Liquid Nitrogen Storage Log Book with appropriate information.

Note: Cell lines in continuous culture are likely to suffer from genetic instability as their passage number increases, as such, subculturecells up to 10-12 passages. More specifically I passaged these cells up to 10 times, and mostly used passage 10 for the isolation. All passage 10 cells were used for the mitochondrial isolation procedure.

The Day Before the Mitochondrial Isolation

Plate cells at the desired seeding density in an appropriate cell culture slide based on the experiment to be performed

On the Day of Mitochondrial Isolation
1) Twenty million cells are needed for the mitochondrial isolation procedure. Ten T75 flasks on day 3 yield the cells needed for the mitochondrial isolation. For H9c2 cells, it usually takes about a few weeks to have ten T75 flasks of the same passage.
2) Lift off all the cells from the P.9 flasks following the regular subculturing protocol. After trypsin is used to detach these cells they will now be the P.10 cells ready to be used for the isolation.

### EXAMPLE 3

### Mitochondrial Isolation

A. Materials
Mitochondrial Isolation Kit for Cultured Cells (ThermoFisher Scientific, Waltham, Mass.)
Halt Protease Inhibitor Cocktail, EDTA free (ThermoFisher Scientific, Waltham, Mass.)
2.0 mL eppendorf tubes
1.5 mL eppendorf tubes
Vortex mixer
Variable-speed-temperature microcentrifuge
70% ethanol
Ice bucket
P-2, P-10, P-20, P-200, and P-1000 micropipetters and pipette tips

B. Protocol: Isolation of Mitochondria using Reagent-Based Method
1) Place the complete growth media in the 37° C. water bath to warm the solution.
2) Turn on the cell culture hood blower (and light if separate switches), and disinfect all the surfaces by spraying them with 70% ethanol and wiping clean. Disinfect each item (such as pipettes, pipette tips, etc.) placed in the cell culture hood by also spraying them with 70% ethanol and wiping clean. Keep the work space in the cell culture hood clean and uncluttered, and keep everything in direct line of sight. Designate a clean side and a waste side for yourself. For a right-handed person the wide, clear space in the center is the work space, the tube rack should be in the rear middle, the reagents and media should be kept on the rear right, the pipettes in the front right, and all waste on the rear left.
3) Change the centrifuge rotor to a microcentrifuge rotor and decrease the temperature to 4° C.
4) Re-suspend the pellet of cells to be subjected to mitochondrial isolation in 1.0 mL growth medium and transfer this solution to a 2.0 mL microcentrifuge tube.
5) Spray an ice bucket and place it inside the cell culture hood.
6) Take out the isolation kit and the protease inhibitor form the freezer and after spraying with 70% ethanol and wiping clean all the reagents, place them in the ice bucket inside the hood.
7) Pellet the harvested cells by centrifuging the cell suspension at ˜850 rcf for 2 minutes. Carefully remove and discard the supernatant.
8) In a 1.5 mL Eppendorf tube (Label A) add 8 μL of Halt Protease Inhibitor to 800 μL of Mitochondrial Isolation Reagent A.
9) Add 800 μL of solution A. Vortex at medium speed for 5 seconds and incubate tube on ice for exactly 2 minutes. Do not exceed the 2 minute incubation.
10) Add 10 μL of Mitochondrial Isolation Reagent B. Vortex at maximum speed for 5 seconds.
11) Incubate tube on ice for 5 minutes, vortexing at maximum speed every minute.
12) In a 1.5 mL Eppendorf tube (Label C1) add 8 μL of Halt Protease Inhibitor to 800 μL of Mitochondrial Isolation Reagent C.
13) Add 800 μL of solution C1. Invert tube several times to mix (do not vortex).
14) Centrifuge tube at 700 rcf for 10 minutes at 4° C.
15) Transfer the supernatant to a new 2.0 mL tube and centrifuge at 3,000 rcf for 15 minutes at 4° C.
16) Transfer the supernatant containing the cytosol fraction to a new tube. The pellet contains the isolated mitochondria.
17) In a new 1.5 mL Eppendorf tube (Label C2) add 5 μL of Halt Protease Inhibitor to 500 μL of Mitochondrial Isolation Reagent C.
18) Add 500 μL of solution C2 to the pellet and centrifuge at 12,000 rcf for 5 minutes. Discard the supernatant.
19) Start labeling the native cell's mitochondria with MitoTracker Green FM.
20) Maintain the mitochondrial pellet on ice at all times during the procedure. Do not attempt to freeze and thaw the pellet as this will compromise mitochondrial integrity.
21) Start labeling the isolated mitochondria with pHrodo Red SE.

### EXAMPLE 4

### Mitochondrial Labeling

A. Materials
PHrodo Red, Succinimidyl Ester (ThermoFisher Scientific, Waltham, Mass.)
MitoTracker® Green FM (ThermoFisher Scientific, Waltham, Mass.)
NucBlue Live ReadyProbes Reagent (Hoechst 3342) (ThermoFisher Scientific, Waltham, Mass.)
Dulbecco's Phosphate Buffered Saline (DPBS(1X)), Calcium, Magnesium
(ThermoFisher Scientific, Waltham, Mass.)
Methyl Cyanide/Acetonitrile (MeCN)
15.0 mL conical tube
1.5 mL eppendorf tube

B. Preparation Protocol

Labeling Native cell's Mitochondria with MitoTracker® Green FM, MitoTracker® Green FM (20×50 ug) Desiccate, protect from light. Store between −25 to −5° C., ThermoFisher Scientific Product#: M7514, Lot #1785959. Excitation/Emission: 490 nm/515 nm; FITC channel (GFP) Mass: 50 ug; Molecular Weight: 671.88 g/mole Stock solution concentration 1 mM; Working solution concentration 625 nM.

To label native mitochondria, cells are simply incubated with MitoTracker® Green FM, which diffuses across the plasma membrane and accumulates in active mitochondria. To make the stock solution of 1 mM, 75 μL (74.418 μL) DMSO is used to dissolve the powder. For the staining a final concentration of 625 nM is desired. Since an 8-wells chamber slide is used with each well holding 0.5 mL media, the volume of the suspension buffer is 4 mL, and 2.5 μL of the stock solution is added to this volume to make the working solution. Phosphate-buffered Saline (PBS) is used to perform the labeling; complete growth media cannot be used since the reduced forms of the MitoTracker probes are susceptible to potential oxidases in serum.

### Calculations:

Stock solution: 1mM; Desired final volume: **4 mL; Desired concentration: 625 nM; Required volume: *2.5 μL.

Labeling Isolated Mitochondria with PHrodo Red SE Label

PHrodo™ Red, succinimidyl ester (PHrodo™ Red, SE) 1 mg Desiccate, protect from light. Store between −25 to −5° C. ThermoFisher Scientific Product #:P36600 Lot #1749822. Excitation/Emission: 566 nm/590 nm; TRITC channel (m-cherry) Mass: 1mg; Molecular weight: 650 g/mole; Stock solution concentration 1 mg/mL ˜1.53 mM concentration.

### Protocol:

The importance of using this dye as opposed to other dyes is that pHrodo™ Red dye conjugates are non-fluorescent outside the cell, but fluoresce brightly red after being endocytosed; the lack of fluorescence outside the cell would serve as clear indicator of mitochondrial internalization through transplantation technique once and if a signal is detected under m-cherry channel. The fluorescence of the novel PHrodo™ Red dye dramatically increase as pH decreases from neutral to the acidic making it an ideal dye to study internalization.

In the written protocol accompanying the product, 150 μL of DMSO is suggested to be used for dissolving the 1 mg powder, to achieve a 10.2mM final concentration. However, the desired concentration was 1 mg/mL and since the powder is 1 mg, 1 mL of the solvent (DMSO) would be needed. It is further suggested to use 1 μL of this stock solution per 1 mL cells with 1 million/mL concentration. In the mitochondrial isolation step, ˜20 million cells are required to carry on the protocol. Hence assuming about 20 million cells are used in each isolation procedure, for the subsequent staining step 20 μL of dye should be used. Since once the PHrodo™ Red SE powder is dissolved in DMSO, it should be used immediately, single-use aliquots are advantageously prepared.

To do so the powder was dissolved in Methyl Cyanide/Acetonitrile (MeCN) and aliquoted into single-use tubes, which was subsequently followed by evaporating off the MeCN to bring it back to powder. The aliquots are stored at −20 C in sealed bags with desiccant packs. When ready to use, 20 μL of DMSO will be added to the powder for a final concentration of 1 mg/mL.

### Procedure:

1) Turn off the hood's light
   Labeling Native cell's Mitochondria with MitoTracker® Green FM
2) In a 15.0 mL conical tube add 2.5 μL of MitoTracker Green FM to **4.0 mL of DPBS.
3) Cover an 8-well glass chamber slide with foil. Remove the media, and add the prewarmed (37° C.) working staining solution.
4) Incubate for a total of 45 minutes.
5) After staining is complete the staining solution is slowly removed from the wells, using a p-200 pipette, as to not stress the cells and prevent detachment.
6) The cells are immediately supplied with 400 μL of fresh pre-warmed media, which is very gently dispersed into the well using a p-1000. This media is removed once more to perform a second wash step and resupplied to the well again.
   **0.5 mL/well×8 wells=4 mL.
      Labeling Isolated Mitochondria with pHrodo Red SE Label
1) In a 1.5 mL eppendorf, resuspend the mitochondrial pellet in 1 mL sterile PBS. Cover the tube with foil.
2) Take a single-use aliquot of pHrodo™ Red and add 20 μL of DMSO to it.
3) Pipette the pHrodo Red SE solution to the mitochondrial suspension. Gently tap with finger to mix and incubate at 4° C. for 30 minutes.
4) (Spin at 12,000 rcf for 5 minutes, and wash with sterile PBS)×2
   Other Dyes we use to Label the Mitochondria to be Transplanted: pHrodo Green STP Ester, CellLight Mitochondria-RFP, BacMam 2.o0
   Labeling the Plasma Membrane with CellMask Deep Red Plasma Membrane Stain
5) Prepare a 1× working solution (by adding 1 uL of the stain to 1 mL PBS) and staining the cells for 10 minutes at 37° C., followed by two wash steps.

### Other Dyes we use to Label Plasma Membrane: CellLight Plasma Membrane-CFP, BacMam 2.0

6) Labeling the Cell Nuclei with NucBlue Live (Hoechst 3342)Although, it is suggested to use 1 drop for every milliliter of media and that only 500 μL is in each well, use 1 drop of NucBlue to label the cell's nuclei.

### EXAMPLE 5

### Co-Incubation of Isolated Mitochondria with Cardiomyocytes

### A. Materials:

Ice bucket

### B. Protocol:

1) Co-incubate these fluorescently-labeled mitochondria in dark with cardiomyocytes for 10 minutes at 4° C. (on top of the ice in the ice bucket).
2) Turn on the IX-83Inverted Motorized Microscope, attach the CO₂ supply and open the Metamorph software during this 10 minutes.

### EXAMPLE 6

### Dynamics of Mitochondrial Internalization Visualized through Time-Lapse Microscopy

### A. Materials:

IX-83Inverted Motorized Microscope (Olympus America, Center Valley, Pa.)

### B. Protocol:

1) Set a 28-hour time-lapse study and instruct the instrument to automatic images at multiple locations every 5 minutes.
2) Label the cell nuclei after the time-lapse study has been completed and acquire the desired number of images for all channels (fluorescence and phase).
3) Analyze the respective data using ImageJ software.
   Other Imaging Instruments we have used to Visualize are:

Zeiss Laser Scanning 780 microscope

Keyence Structured Illumination microscope

Nanolive Holotomography microscope

Nikon's CSU-W 1 SoRa

Nikon's N-SIM

### Outcome:

Based on the endosymbiosis theory of mitochondrial origin, it was hypothesized and tested whether mitochondrial transplantation into cardiomyocyte is feasible. This was the first step in the attempt to make available a novel cellular biotherapy for mitochondrial cardiomyopathy patients, who suffer from a condition in which their heart-muscle structure, function, or both is abnormal due to genetic defects involving the mitochondrial respiratory chain.

Mitochondrial internalization was observed at various times during our 28-hour time-lapse study, with the shortest being a little past 4 hours. The internalized mitochondria remained present in cardiomyocytes for the 28-hour duration of the study (FIG. 2).

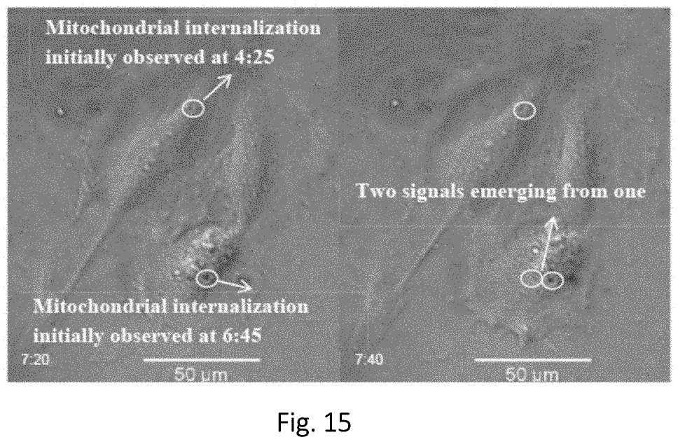

Furthermore, there were instances of emergence of two red signals from one, which is believed may represent the fission process. However, it is also possible that signal was originated from two separate mitochondria in close proximity, which only became distinct at a later point when they had moved away from one another (FIG. 15).

To determine the success of mitochondrial transplantation the pHrodo Red SE signal was relied on, which as a pH dependent internalization marker that would only be expected to fluoresce once inside the cell and not outside the cardiomyocyte. To ensure that the red fluorescence signal observed in the transplantation experiment indeed roots from the internalization of the isolated-labeled mitochondria and not the passive diffusion of the dye by itself, a set of control experiments were performed with four groups: 1) no cell +pHrodo Red 2) cells only, no dye 3) cells+pHrodo Red, 4) cells+PHrodo Red and Mito Tracker Green FM. In the control experiment the isolated mitochondria were eliminated and in the co-incubation step only pHrodo Red SE is co-incubated with cardiomyocytes and of course the wash step is also eliminated. No red fluorescence was detected in the control groups (FIG. 8). Therefore, it was successfully shown that the isolated-labeled mitochondria are actively endocytosed into the cell, and that the pHrodo Red SE dye by itself cannot passively get internalized.

It was demonstrated that mitochondrial transplantation is possible and through this application, it is aimed to study whether post-transplanted cells with mitochondrial dysfunction exhibit improved bioenergetics and contractibility. It is also planned to follow up the study by determining whether the transplanted mitochondria are naturally adopted by the host cardiomyocyte.

### EXAMPLE 7

### Adoption of Transplanted Mitochondria

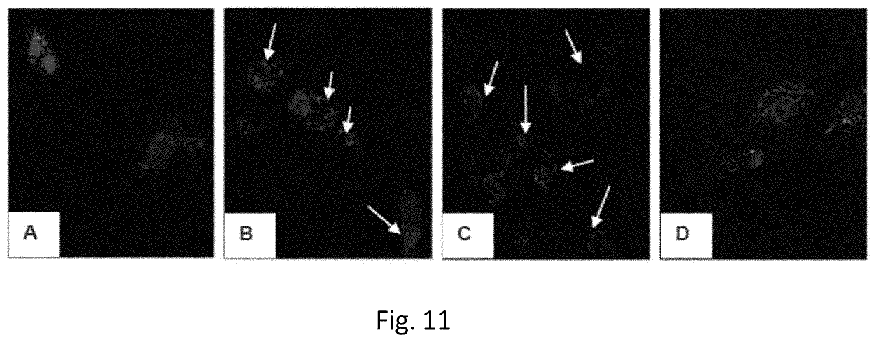

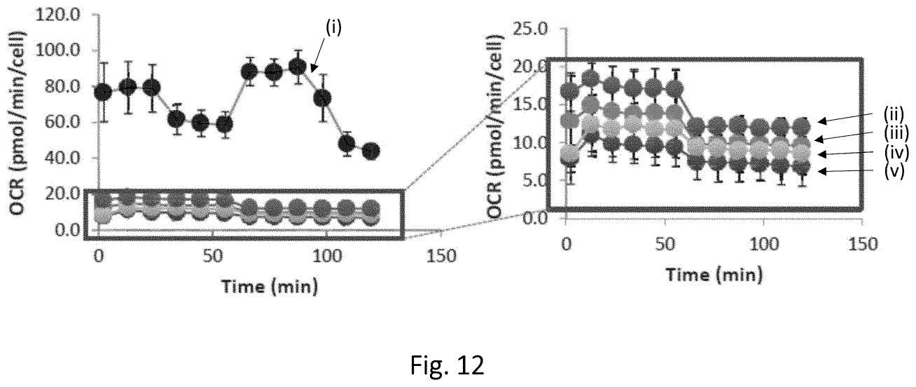

To check whether the transplanted mitochondria are adopted by the host and lead to any functional gain, rho zero cells were monitored post-mitochondrial transplantation. Rho zero cells are mitochondrially-depleted cells that have small fragments of mt-DNA but as a result of mt-DNA mutation have non-functional mitochondria and as such do not perform oxidative phosphorylation, and oxidative phosphorylation is much more efficient than glycolysis. To meet their metabolic needs, rho zero cells need to be grown on media supplemented with uridine. Hence, to study whether mitochondrial transplantation leads to functional gains, mitochondria (from five million rat L6 skeletal cell) were transplanted into (human ARPE-19 retinal) rho zero cells and the media was changed from rho zero media to that of WT lacking the uridine supplement either at the time of transplantation or 24 hours later, and were looked under the microscope 48-hour post transplantation to look at the profile of dead versus live cells. Compared to the rho zero cells in WT media, the rho zero cells that had received mitochondrial transplant treatment had a better survival (FIG. 11). Although we had suspected that having the uridine supplement during the initial hours of transplantation might help reduce the rho zero cell's environmental stress and lead to better mitochondrial uptake and hence bioenergetics, there seems to be no significant difference between removing the uridine supplement at the time of transplantation versus 24 hours later. Moreover, in bioenergetics experiments using Seahorse XF24 Analyzer, transplantation of autologous mitochondria (from five million ARPE-19 WT cells into 50K ARPE-19 rho zero cells) improved the basal respiration by almost two folds (FIG. 12). The experiment was performed in quadruplicates and the data was normalized to protein level using Bradford reagent.

### EXAMPLE 8

Short-Term and Long-Term Bioenergetics of Mitochondrial Transplantation into Cardiomyocytes

Seahorse Extracellular Flux Analyzer is an assay used to measure the cellular oxygen consumption rate and extracellular acidification rate as measurements of cell/mitochondrial metabolism. To find the optimized settings, cells are first seeded at different densities to find the optimized cell seeding density, followed by different concentrations of the drug compound (FCCP). The cells are plated in at least triplicate at the optimal cell concentration, and the Oxygen Consumption Rate (OCR) and Extracellular Acidification Rate (ECAR) will be determined. The cells' metabolic rates are measured after sequential treatment with three compounds: Oligomycin, FCCP, and Rotenone/Antimycin A.

Oligomycin is a phosphorylation inhibitor that binds to a polypeptide (OSCP) in the Fₒ baseplate and blocks ATP synthesis by the Fₒ/F₁ ATPase. FCCP (p-trifluoromethoxy carbonyl cyanide phenyl hydrazone) is an uncoupler that shields the electric charge as the ion passes through the membrane, providing a polar environment and increasing proton permeability. Rotenone is a respiration inhibitor that blocks NADH dehydrogenase (complex I). Antimycin is a respiration inhibitor that blocks complex III.

The data is analyzed using Seahorse XF Software, and normalization is done by cell protein levels quantitated using Micro BSA reagent (ThermoFisher Scientific, Waltham, Mass.). A welch's test will be employed for mean values, and results with p<0.05 will be considered as statistically significant.

We hypothesized and tested whether the internalization of the transplanted non-autologous mitochondria leads to enhanced bioenergetics, by conducting Cell Mito Stress Test using a Seahorse XF24 Flux Analyzer. We measured the oxygen consumption rate (OCR), which represent the rates of oxidative phosphorylation and extracellular acidification rate (ECAR), which denotes the summation of acid (H+) produced from glycolysis and the TCA cycle (via CO2), when obtained through Mito Stress Test. Subsequently, we compared the results to the control groups with no transplantation event. Baseline and post-transplant rates were measured at four different time points: post 2-day to measure acute effects (FIG. 3), post 7-day, post 14-day to check bioenergetic indices around mitochondria turnover, and finally post 28-day to check for long-term effect (FIG. 4).

We observed an upward shift in OCR kinetics 2-day post-mitochondrial transplantation compared to the baseline (FIGS. 3). Compared to the control, we observed a statistically significant improvement in basal respiration and ATP production 2-day post-transplantation (p=0.031 and p=0.025, respectively; FIG. 3). Maximal respiration and spare respiratory capacity—the cell's bioenergetics reserve in meeting a situation of high demand or acute/chronic stress—were also enhanced post 2-day, although not statistically-significantly, p=0.060 for maximal respiration and p=0.080 for spare respiratory capacity (FIG. 3). Coupling efficiency was unchanged as both transplanted and native mitochondria were healthy (FIG. 3).

Based on our bioenergetics results, it appears that in the normal cells, mitochondrial transplantation leads to acutely enhanced bioenergetics. However, these initial effects seem to diminish over time and return to that of the baseline control (FIG. 4).

In addition to OCR, we evaluated the ECAR from the Mito Stress Test (FIG. 3 and FIG. 4). In many cell types, mitochondrial activity (TCA cycle) that leads to CO2 production is a significant source of extracellular acidification (Mookerjee S A, Goncalves R L S, Gerencser A A, Nicholls D G, Brand M D. The contributions of respiration and glycolysis to extracellular acid production. Biochimica et Biophysica Acta (BBA)-Bioenergetics. 2015; 1847:171-181). Since in our observation the ECAR significantly decreased after R+A injection, we suppose that in our samples the acidification was generated through the TCA cycle. However, from Mito Stress Test, only qualitative interpretation of the changes in ECAR data can be made for the kinetics after the injection of rotenone and antimycin. If the ECAR significantly decreases (i.e., low residual ECAR) in response to R+A, we infer the ECAR (prior to the injection of R+A) is primarily being generated via CO₂ production from the TCA cycle. If the ECAR remains constant and/or increases in response to R+A, then the ECAR prior to R+A treatment is primarily being generated via glycolysis (Divakaruni A S, Paradyse A, Ferrick D A, Murphy A N, Jastroch M. Chapter sixteen—analysis and interpretation of microplate-based oxygen consumption and pH data. In: Murphy A N, Chan D C, eds. Methods in enzymology. Academic Press; 2014:309-354). The exogenous pyruvate and glutamine supplemented in the media can be directly shunted into the TCA cycle, which lead to CO₂ production independent of the CO₂ produced from the pyruvate generated from glucose through glycolysis. Since we observed a decrease in response to R+A in both control and transplant groups, we infer that that CO₂ is primarily being generated via TCA cycle in this cell type. Another viewpoint indicates that in both control and transplant groups, the cells were burning through the exogenous pyruvate and glutamate. However, on the contrary, we interpret our ECAR data (FIGS. 3 and 4), such that the increase in ECAR kinetics in transplant vs. the control group (FIG. 3) is due to CO₂ production from the increased TCA cycle in transplant group. Glycolytic Rate Assay can delineate the sole contribution of glycolysis or CO2 to ECAR, which we did not perform in the current experiments.

Based on our observations, the enhanced bioenergetics state is temporary, which may imply that either the cells' mitochondrial content is returned to physiological levels, or the overall mitochondrial activities are reduced to reflect the cell's bioenergetics needs. Since the spare respiratory capacity, which measures the cell's capacity in meeting a situation of high energy demand, is not significantly changed in the transplant vs. the control, it is less likely that the overall mitochondrial activities have dampened and more likely that mitochondrial content has returned to physiological levels. Our observation corroborates with the findings of Finck and Kelly, and accordingly, we hypothesize that a normal cell clears any excess mitochondria keeping the mitochondrial content at physiological levels, as the plausible mechanism for the return of the bioenergetics indices to the baseline levels in long-term. In case of an adjustment of the mitochondrial content, it is yet unclear whether the newly transplanted mitochondria is cleared from the cell or if the transplanted mitochondria have been adopted by the host and the total mitochondrial level regardless of the recipient or donor status is reduced to bring the bioenergetics levels back to physiological state. We aim to answer this question through a time-course mitochondrial DNA sequencing experiment post-transplantation.

Moreover, the long-term bioenergetics profile suggests that there is no negative bioenergetic consequences due to mitochondrial transplantation and that the intervention can be considered safe for the cell. The question remained to be answered is whether cells with damaged mitochondria would take advantage of the newly introduced mitochondria or not, as those cells could uptake and adopt the mitochondria differently than a normal cell, which is already able to efficiently meet its bioenergetics demands.

### EXAMPLE 9

### Mitochondrial Superoxide Production Post-Mitochondrial Transplantation

In a normal tightly coupled electron transport chain, approximately 1-3% of the consumed oxygen is incompletely reduced, which can lead to superoxide production as a result of the interaction between the leaky electrons and molecular oxygen (Jastroch M, Divakaruni A S, Mookerjee S, Treberg J R, Brand M D. Mitochondrial proton and electron leaks. Essays Biochem. 2010; 47:53-67; and Batandier C, Fontaine E, Kériel C, Leverve X M. Determination of mitochondrial reactive oxygen species: Methodological aspects. Journal of Cellular and Molecular Medicine. 2002; 6:175-187). Since we had observed a statistically-significant enhancement in bioenergetics due to increased oxidative phosphorylation at 2-day post-transplantation, we used the mitochondrially-targeted derivative of hydroethidine (HE)—MitoSOX Red—to detect and quantitate the level of superoxide produced by mitochondria in post-transplant and control groups 2-day after transplantation. Compared to control, no statistically-significant difference was found in superoxide production in the post-transplant group (p=0.99 and p=0.83 for set 1 and 2 transplantations, respectively; FIG. 5). The reported superoxide production was not assessed for the same Seahorse experiment, but a representative one.

Mitochondrial superoxide production was measured 2-day post transplantation using MitoSOX Red in control and transplant groups, and no significant difference was observed among the groups (FIG. 5).

### EXAMPLE 10

### Mitochondrial Function Declines with Increasing Passage Number (Aging)

We observed a reduction in mitochondrial function in the studied groups with increasing the passage number. ATP production and coupling efficiency, positive indicators of bioenergetics, significantly decreased with increasing passage number, while proton leak, a negative indicator of bioenergetics, significantly increased (FIG. 6). This observation corroborates with the findings of a previous study by Witek et al. (Witek P, Korga A, Burdan F, Ostrowska M, Nosowska B, Iwan M, Dudka J. The effect of a number of h9c2 rat cardiomyocytes passage on repeatability of cytotoxicity study results. Cytotechnology. 2016; 68:2407-2415), where they observed an increase in oxidative stress in cells with passaging of cardiomyocytes. It appears that mitochondrial function is reduced in cells with higher passage number and thus these cells are not representative of the initial passages.

### EXAMPLE 11

### Fluorescence Lifetime Imaging (FLIM) to Evaluate the Post-Transplantation Bioenergetics on a Single Cell Level

In FLIM the lifetime of a fluorophore, or in other words the time between the excitation and emission is found. FLIM (using the time domain) may be reserved as a potential additional method to characterize metabolic state and oxidative stress using phasor analysis of endogenous biomarkers in evaluating bioenergetics on the level single cell. More specifically, to study metabolism, absorbance measurements will be taken of free NADH and bound NADH for the control as a reference to mark the phasor lifetime on the plot. In its free state due to self-quenching, the lifetime of NADH is about 0.4 ns, which is significantly lower than that of the bound state, which is why FLIM can easily differentiate between free and protein bound forms of NADH. Two-photon laser microscope set to 740 nm may be used to excite free and bound NADH, and the fluorescence emission is collected using a Photomultiplier tube with a BP filter 420-500 nm. The raw intensity data is transformed into polar coordinates by plotting sine and cosine. Similarly, oxidative stress is assessed by looking at long lifetime species (LLS) which in oxidative stress is characterized by clustering in the lifetime phasor plot.

### EXAMPLE 12

### Supper-Resolution Microscopy

To overcome the limitation posed by the diffraction limit of the light and to characterize the nanoscale mitochondria, CSU-W1 SoRa, N-SIM, or Direct Stochastical Optical Reconstruction Microscopy (dSTORM) and Stimulated Emission Depletion Microscopy (STED) may be used. In conventional light focused microscopy, the objective focuses the light to a point, but because light propagates as a wave it is not possible for the lens to focus all of the light into a single point and the light will be smeared, which gives to the diffraction limit of light (˜200 nm) which is explained by D=λ/2NA. In supper-resolution microscopy not all the fluorophores are excited at the same time, which allows for the detection of the features within this zone at different times to resolve the hidden details.

Super-resolution microscopy will enable us to characterize the fission and fusion dynamics, and parameters such as but not limited to propensity and rate of fission, fusion, mitophagy, and biogenesis, the average mitochondria uptake time, mitochondrial turnover, etc. which can be used to mathematically model and inform decisions on the treatment regimen, dosage and frequency of dosage.

### EXAMPLE 13

### Xenogeneic Mitochondrial Transplantation to Disturb Bioenergetics of T Cell Leukemia Cell Lines

We have been focused on feasibility of mitochondrial transplantation and have recently shown that cellular bioenergetics are enhanced short-term after mitochondrial transplantation in normal rate cardiomyocytes. As well, through our experiments, we have shown that non-autologous mitochondrial transplantation between the two rat cell lines, and interspecies mitochondrial transplantation between rat and human cell lines are possible.

The fact that we could gain control over the cells' bioenergetics (short-term supercharge [positive bioenergetic effect]) after non-autologous mitochondrial transplantation provide a basis to test modulation of the bioenergetics of neoplastic cells (negative bioenergetic effect)

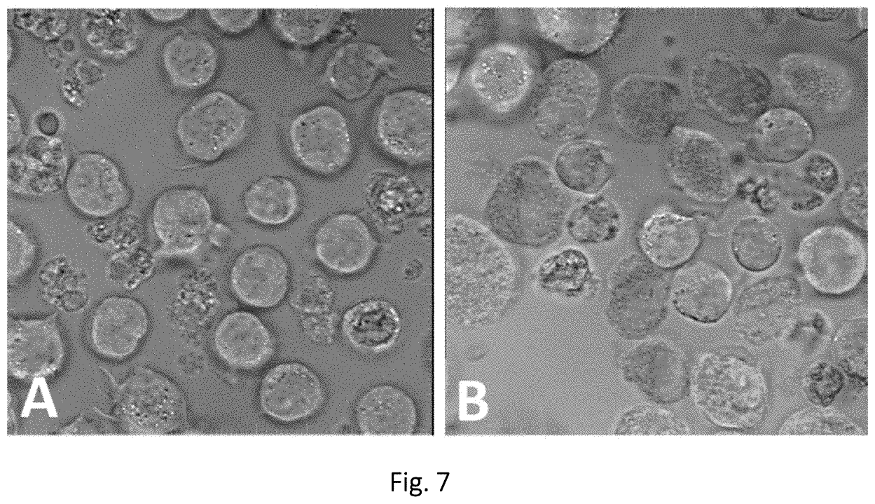

We have shown the feasibility of non-autologous transplantation of NHDF-Neo human fibroblasts into an immortalized line of human T lymphocyte (Jurkats) (FIG. 7). This study provides a basis for gaining control over the neoplastic cells' bioenergetics through transplantation of non-autologus or xenogeneic mitochondria into an immortalized line of human T lymphocyte.

During the past decades, many approaches have been taken to eradicate the neoplastic cells. Classic chemotherapy agents, either alone or in combination with other drugs, target cells at different phases of the cell cycle: (1) Alkylating agents damage the neoplastic DNA; (2) Antimetabolites interfere with DNA and RNA by replicating RNA ‘sand DNA's nucleotides; (3) Anti-tumor antibiotics, Topoisomerase inhibitors, and Mitotic inhibitors alter DNA inside the neoplastic cells to keep them from growing and multiplying. Newer approaches include: Targeted chemotherapy that takes advantage of differences between normal and neoplastic cells, and Immunotherapy that boosts or alter patient's immune system. However, to the best of our knowledge, eliminating or debilitating the neoplastic cells by means of corrupting their bioenergetics via manipulating their mitochondria has not yet been attempted.

We demonstrated that mitochondrial transplantation into Jurkat cells is feasible. Jurkat cells are an immortalized line of human T lymphocyte cells that are used to study acute T cell leukemia. Mitochondrial transplantation from NHDF-Neo (Human Dermal Fibroblasts) to Jurkat cells was successfully performed through coincubation. Isolated mitochondria labeled with pHrodo Red are shown in FIG. 7.

To study the bioenergetics of Jurkat cells after non-autologous mitochondrial transplantation Seahorse XF24 Flux Analyzer will be used to measure the OCR and ECAR, which represent the rates of oxidative phosphorylation and glycolysis, respectively. The results will be compared to the control Jurkat cells with no transplantation. Baseline and post-transplantation rates are measured at four different time points: 2-days, 7-days, 14-days and 28-days post-transplant to test how Jurkat cells' bioenergetics are affected by non-autologous mitochondrial transplantation. Non-autologous mitochondrial transplantation disturbs the Jurkat cells' bioenergetics compared to a control group with no transplantation event.

To study the Jurkat cells' susceptibility to cyclophosphamide, doxorubicin, vincristine and etoposide after mitochondrial transplantation, transplanted cells are compared with control Jurkat cells. A polydimethylsiloxane (PDMS)-based microfluidic device with a bypass channel around a droplet formation well, as previously reported by others (Boukellal H, Selimović Š, Jia Y, Cristobal G, Fraden S. Simple, robust storage of drops and fluids in a microfluidic device. Lab on a Chip. 2009; 9:331-338; and Wong A H-H, Li H, Jia Y, Mak P-I, Martins R Pd S, Liu Y, Vong C M, Wong H C, Wong P K, Wang H, Sun H, Deng C-X. Drug screening of cancer cell lines and human primary tumors using droplet microfluidics. Scientific Reports. 2017; 7:9109-9109) is used for on chip drug screening using cyclophosphamide, doxorubicin, vincristine and etoposide (CHOEP protocol, Pfreundschuh M, Trümper L, Kloess M, Schmits R, Feller AC, Rübe C, Rudolph C, Reiser M, Hossfeld D K, Eimermacher H, Hasenclever D, Schmitz N, Loeffler M, the German High-Grade Non-Hodgkin's Lymphoma Study G. Two-weekly or 3-weekly chop chemotherapy with or without etoposide for the treatment of elderly patients with aggressive lymphomas: Results of the nhl-b2 trial of the dshnhl. Blood. 2004; 104:634-641; and Pfreundschuh M, Trümper L, Kloess M, Schmits R, Feller A C, Rudolph C, Reiser M, Hossfeld D K, Metzner B, Hasenclever D, Schmitz N, Glass B, Rübe C, Loeffler M, the German High-Grade Non-Hodgkin's Lymphoma Study G. Two-weekly or 3-weekly chop chemotherapy with or without etoposide for the treatment of young patients with good-prognosis (normal ldh) aggressive lymphomas: Results of the nhl-bl trial of the dshnhl. Blood. 2004; 104:626-633). For on chip assays, the number of cells is counted in brightfield and red fluorescence images and cell viability is calculated according to Wang et al. (Wong A H-H, Li H, Jia Y, Mak P-I, Martins R Pd S, Liu Y, Vong C M, Wong H C, Wong P K, Wang H, Sun H, Deng C-X. Drug screening of cancer cell lines and human primary tumors using droplet microfluidics. Scientific Reports. 2017; 7:9109-9109). Through these assays, the effect of mitochondrial transplantation on Jurkat cells' susceptibility to CHOEP is quantified, as a standard chemotherapy regimen. The results of this study provide a basis for the application of mitochondrial transplantation in cancer biotherapy.

### EXAMPLE 14

### Intracellular Consequences of Mitochondrial Transplantation in Induced Pluripotent Stem Cell-Derived Cardiomyocytes

In one embodiment, the intracellular consequences of mitochondrial transplantation in human induced Pluripotent Stem Cell (iPSC)-derived cardiomyocytes can be considered. This concept can be a basis for mitochondrial transplantation in iPSC-derived cardiomyocytes in a variety of patients with congenital and acquired myocardial diseases.

According to the Centers for Disease Control and Prevention (CDC), in the United States alone, heart failure (HF) affects 5.7 million individuals and annually costs the nation over $30 billion, which includes the cost of health care services, medications, and missed workdays (Roger V L, Go A S, Lloyd-Jones D M, Benjamin E J, Berry J D, Borden W B, Bravata D M, Dai S, Ford ES, Fox C S, Fullerton H J, Gillespie C, Hailpern S M, Heit J A, Howard V J, Kissela B M, Kittner S J, Lackland D T, Lichtman J H, Lisabeth L D, Makuc D M, Marcus G M, Marelli A, Matchar D B, Moy C S, Mozaffarian D, Mussolino M E, Nichol G, Paynter N P, Soliman E Z, Sorlie P D, Sotoodehnia N, Turan T N, Virani S S, Wong N D, Woo D, Turner M B. Heart disease and stroke statistics-2012 update: A report from the american heart association. Circulation. 2012; 125:e2-e220). Most HF cases are due to acquired and some due to congenital cardiomyopathies. Patients with acquired cardiomyopathies usually present with a history of myocardial infarction (MI) or objective evidence of coronary artery disease (CAD) and are usually classified to have ischemic cardiomyopathy (Felker G M, Shaw L K, O'Connor C M. A standardized definition of ischemic cardiomyopathy for use in clinical research. Journal of the American College of Cardiology. 2002; 39:210), whereas congenital cardiomyopathies occur as a result of abnormalities in cardiomyocytes, usually due to a gene mutation. Regardless of the cause, mitochondrial impairment plays an important role in all types of cardiomyopathies. Mitochondrial respiratory chain is responsible for much of the cellular energy production. Since cardiomyocytes require high energy for their dynamic function, changes in mitochondrial respiration and function have been commonly reported in different models of pathological cardiac remodeling (Schwarzer M, Schrepper A, Amorim P A, Osterholt M, Doenst T. Pressure overload differentially affects respiratory capacity in interfibrillar and subsarcolemmal mitochondria. American Journal of Physiology-Heart and Circulatory Physiology. 2012; 304:H529-H537; and Zoll J, Monassier L, Gamier A, N'Guessan B, Mettauer B, Veksler V, Piquard F, Ventura-Clapier R, Geny B. Ace inhibition prevents myocardial infarction-induced skeletal muscle mitochondrial dysfunction. Journal of Applied Physiology. 2006; 101:385-391).

We have demonstrated that cellular bioenergetics are enhanced short-term after mitochondrial transplantation in normal rate cardiomyocytes. While mitochondrial transplantation is an exciting recent achievement in the modern history of medicine, not much is known about its intracellular consequences. In particular, it is unknown whether the host cardiomyocytes adopts the transplanted mitochondria' s mtDNA and whether the positive effect of mitochondrial transplantation on bioenergetics of cardiomyocytes with defective mitochondria is permanent.

In one embodiment we will test the hypothesis that the mtDNA of mitochondria transplanted into iPSC-derived cardiomyocytes from patients with mitochondrial disease is naturally adopted by the host and enhances the host's cellular bioenergetics. Additionally, we plan to determine the transplanted mitochondria's survival, and functional interactions among transplanted and host mitochondria with the nucleus.

In one embodiment, we will determine whether the normal human mitochondria transplanted into iPSC-derived cardiomyocytes of patients with mitochondrial disease are naturally adopted via sequencing the mtDNA post-transplantation through time.

In one embodiment, we will test whether transplanting healthy mitochondria into iPSC-derived cardiomyocytes with mitochondrial defects leads to permanent improved cell bioenergetics.

In one embodiment, we will identify the differentially-expressed genes and pathways involved through RNA-Seq analysis of the iPSC-derived cardiomyocytes of patients with mitochondrial disease post-mitochondrial transplantation. Additionally, we will follow up the transcriptomic studies with proteomic studies.

Mitochondrial dysfunction is commonly observed in cardiomyopathies, which can be due to ischemic damage or genetic mutations (Meyers D E, Basha H I, Koenig M. Mitochondrial cardiomyopathy: Pathophysiology, diagnosis, and management. Texas Heart Institute Journal. 2013; 40:385-394). Mitochondria are the evolved autotrophic bacteria that were engulfed by the eukaryotic cells over two billion years ago. These micro-organelles possess their own DNA (mtDNA), which encodes most of their components and defines a genetic system partially distinct from the cell's nuclear genome. Mitochondria are responsible for cellular energetics, and their malfunction leads to cellular injury and eventually cell death. Due to recent advances in genetics, overall, hundreds of acquired and inherited diseases have been discovered that are believed to be due to mitochondrial dysfunction. A prolonged period of ischemia can permanently damage the ability of cardiomyocytes' mitochondria to generate ATP (Pagliaro P, Femmino S, Popara J, Penna C. Mitochondria in cardiac postconditioning. Frontiers in Physiology. 2018; 9:287; and Piper H M, Noll, T., Siegmund, B. Mitochondrial function in the oxygen depleted and reoxygenated myocardial cell. Cardiovasc Res. 1994; 28:1-15). Currently, there is no remedy for acquired or inherited mitochondrial cardiomyopathies.

In one embodiment, we will quantify how host iPSC-derived cardiomyocytes adopt transplanted mitochondria, and identify the inter-mitochondrial and retrograde signaling mechanisms and how transplanted mitochondria improve the performance of human iPSC-derived cardiomyocytes. Identifying the intracellular consequences of mitochondrial transplantation in human iPSC-derived cardiomyocytes provides a basis for evaluating and optimizing mitochondrial transplantation, as a cellular biotherapy for patients with cardiomyopathy.

Identification of differentially-expressed genes and pathways involved, as well as proteins (which are most likely those involved in inter-mitochondrial signaling and mitochondrial retrograde signaling with the nuclei) may elucidate whether mitochondrial transplantation changes cells' transcriptomics and proteomics.

We anticipate the possibility of the presence of post-mitochondrial transplantation mtDNA hybridization between the host and transplanted mitochondria. The experiments are designed so as to either reject or prove the possibility of formation of hybrid mtDNA from two distinct populations of host and transplanted mitochondria in human cardiomyocytes.

In one embodiment, we will measure and compare the bioenergetics of the iPSC-derived cardiomyocytes of patients with mitochondrial disease prior to and post-mitochondrial transplantation. These studies will provide a basis for whether the transplantation of healthy mitochondria into human cells with defective mitochondria, leads to permanent improvement of cell bioenergetics.

While a few groups worldwide have independently shown the feasibility of mitochondrial transplantation, little is known about the intracellular consequences of mitochondrial transplantation. It is possible that mitochondrial transplantation changes a cells' transcriptomics and proteomics.

In some embodiment, we will investigate the intracellular consequences of mitochondrial transplantation in iPSC-derived cardiomyocytes of patients with mitochondrial disease. The experiments will test whether: (1) transplanted normal human mitochondria into iPSC-derived cardiomyocytes from patients with mitochondrial disease are naturally adopted; and (2) transplantation of healthy mitochondria into the iPSC-derived cardiomyocytes with mitochondrial defects lead to improved cell bioenergetics. These studies can provide a basis for using the mitochondrial transplantation process as a cellular biotherapy to mitigate congenital and acquired myocardial diseases with mitochondrial involvement.

iPSC from patients with mitochondrial cardiomyopathy: iPSC lines of patients from a biorepository of patient-specific and de-identified human iPSC lines are utilized. The lines are created by reprogramming patient samples, as described by Burridge et al (Burridge P W, Diecke S, Matsa E, Sharma A, Wu H, Wu J C. Modeling cardiovascular diseases with patient-specific human pluripotent stem cell-derived cardiomyocytes. Methods in molecular biology (Clifton, N.J.). 2016; 1353:119-130). The SCVI Biobank recently recruited patients with mitochondrial disease due to mtDNA mutations, including Myoclonic epilepsy with ragged-red fibers (MERRF), Mitochondrial encephalomyopathy, lactic acidosis, stroke-like episodes (MELAS), and Partial mitochondrial complex I deficiency.

Differentiation of iPSCs to cardiomyocytes: The PSC Cardiomyocyte Differentiation Kit by Thermo Fisher Scientific is used to create cardiomyocytes from the iPSC lines. The kit allows efficient differentiation as characterized by contracting in culture while expressing essential markers, e.g., TNNT2, Nkx2.5, MYH6, and a-actinin. Once beating is detected, the cells are refined in a cardiomyocyte enrichment medium with lactate instead of glucose, which permits selective survival of cardiomyocytes (Herron Todd J, Rocha Andre Monteiro D, Campbell Katherine F, Ponce-Balbuena D, Willis B C, Guerrero-Serna G, Liu Q, Klos M, Musa H, Zarzoso M, Bizy A, Furness J, Anumonwo J, Mironov S, Jalife J. Extracellular matrix—mediated maturation of human pluripotent stem cell—derived cardiac monolayer structure and electrophysiological function. Circulation: Arrhythmia and Electrophysiology. 2016; 9:e003638; and Tohyama S, Hattori F, Sano M, Hishiki T, Nagahata Y, Matsuura T, Hashimoto H, Suzuki T, Yamashita H, Satoh Y, Egashira T, Seki T, Muraoka N, Yamakawa H, Ohgino Y, Tanaka T, Yoichi M, Yuasa S, Murata M, Suematsu M, Fukuda K. Distinct metabolic flow enables large-scale purification of mouse and human pluripotent stem cell-derived cardiomyocytes. Cell Stem Cell. 12:127-137). Flow cytometry is used to quantify the percentage of Troponin T-positive cells to verify the differentiation success. Patient-derived and control cardiomyocytes is used for the mitochondrial transplantation studies.

Statistical analysis for mtDNA analysis: We test if mtDNA hybridization can occur after mitochondrial transplantation. We sequence the mtDNA of the host and transplant cell lines before the transplant event as described above. Based on the acquired mtDNA sequences, we use a statistical plan to be able to distinguish the hybrid mtDNA from the host and transplant mtDNA. Heteroplasmy in mtDNA sequences is possible and may lead to interpretational oversight. To mitigate this, we use implement the statistical plan using a Bayesian framework (Egeland T, Salas A. A statistical framework for the interpretation of mtdna mixtures: Forensic and medical applications. PLoS ONE. 2011; 6:e26723).

Power analysis and statistical methods for Seahorse experiments: Our studies find the minimum relative differences in bioenergetics Seahorse can significantly detect, and the minimum number of well replicates needed. We utilize the method described by Yépez et al. (Yépez VA, Kremer L S, luso A, Gusic M, Kopajtich R, Koňaříková E, Nadel A, Wachutka L, Prokisch H, Gagneur J. Ocr-stats: Robust estimation and statistical testing of mitochondrial respiration activities using seahorse xf analyzer. PLoS ONE. 2018; 13:e0199938). We use the number of wells of the repeated biological samples to 4, 6, 8, 10, 12, 14, and 16 wells on each plate, and use the OCR-Stats algorithm (Yépez V A, Kremer L S, luso A, Gusic M, Kopajtich R, Koňaříková E, Nadel A, Wachutka L, Prokisch H, Gagneur J. Ocr-stats: Robust estimation and statistical testing of mitochondrial respiration activities using seahorse xf analyzer. PLoS ONE. 2018; 13:e0199938) as well as statistical testing. Assuming three plates per comparison and 16 wells per plate, these standard deviations allow the detection of relative differences of 10% to 15% depending on the considered log OCR ratios differences for significance level of 5%.

For statistical analysis, an unpaired two-tailed unequal variance t-test (Welch's Test) is used for the mean values of Seahorse bioenergetics data, once normality is checked using qq plots in R. Results with p<0.05 is considered as statistically significant.

Statistical analysis of proteomics data: Reduction in the data dimension is a central step in processing proteomics data due to the sparsity of significant features in big datasets. Overall proteomics data analysis benefits from feature selection to have its dimensionality of the data reduced. Features are selected or extracted based on uni- and multi-variate methods. Significant features generate a proteomics signature, which can be predictive (Lualdi M, Fasano M. Statistical analysis of proteomics data: A review on feature selection. Journal of Proteomics. 2019; 198:18-26).

We determine whether the normal human mitochondria transplanted into iPSC-derived cardiomyocytes of patients with mitochondrial disease are naturally adopted via sequencing the mtDNA post-transplantation through time.

An unanswered question from our bioenergetics studies is whether the return of mitochondrial bioenergetics to original physiologic levels was because the total mitochondrial content was reduced regardless of the source (i.e., transplant or host) or if the newly transplanted mitochondria were exclusively cleared from the cells. We address this question through time-course mtDNA sequencing. If the transplant mtDNA persist in post-transplant cells through time, that means that the host has adopted the transplanted mitochondria. We also investigate whether a new population—hybrid mtDNA of the host and transplant—arises due to the mitochondrial dynamics of fission and fusion between the transplant and host mitochondria. Gilkerson et al. (Gilkerson R W, Schon E A, Hernandez E, Davidson M M. Mitochondrial nucleoids maintain genetic autonomy but allow for functional complementation. The Journal of Cell Biology. 2008; 181:1117-1128) suggests that mitochondria can exchange nucleoids through fusion and fission;however, the nucleoids themselves maintain genetic autonomy.

To track the changes in mtDNA profile, the iPSC from subjects with different haplotypes are used (i.e., haplotype from an individual with African background and another individual from Euro-Indian or East-Asian background).

Since we have reached the ability to isolate the mitochondrial organelle, our main approach to isolating the mtDNA needed for sequencing is to directly extract mtDNA from the isolated mitochondria post-transplantation. This prevents us from losing the information in the primer region if primers were to be used to PCR amplify the mtDNA. However, if the yield is not sufficient for sequencing, we isolate the total DNA and amplify the mtDNA through PCR amplification.

We expect that transplanting healthy mitochondria into iPSC-derived cardiomyocytes with mitochondrial defects leads to permanent improved cell bioenergetics.

Based on our results, mitochondria transplantation into normal cardiomyocytes leads to transient positive effects in bioenergetics. However, it is not yet known how dysfunctional cardiomyocytes would respond to healthy transplanted mitochondria. In some cases, bioenergetics enhancement may be used long-term after mitochondrial transplantation in iPSC cardiomyocytes from patients with mitochondrial disease.

To investigate the bioenergetics consequences of mitochondrial transplantation in iPSC-derived cells from patients with mitochondrial dysfunction, oxygen consumption rate (OCR), extracellular acidification (ECAR) rates, and ATP content are measured pre- and post-transplantation using a Seahorse XF24 Flux Analyzer, after optimizing the cell seeding density and injection drug concentrations. Raw data are normalized to total protein using a micro BCA protein assay. We anticipate the results to inform us of whether mitochondrial transplantation in iPSC-derived cardiomyocytes from patients with mitochondrial disease will have long-term positive bioenergetics effects.

Identification of differentially-expressed genes and pathways involved is done through RNA-Seq analysis of the iPSC-derived cardiomyocytes of patients with mitochondrial disease post-mitochondrial transplantation. By way of the RNA-Seq analysis of the samples in a time-course study, we will investigate genes that are differentially expressed, and the pathways and gene networks involved. Even though mitochondria were autonomous—similar to bacteria—billions of years ago, through evolution and in time, they became semi-autonomous, with some of their genes being translocated into the nucleus, which the mitochondria rely on. As such, we hypothesize that if the recipient cells permanently adopt the transplanted mitochondria, there will be an increased expression of the genes involved in the retrograde and anterograde signaling. We also expect the other mitochondrial dynamics to be affected. For instance, the transcripts involved in mitophagy and apoptosis in combination with the mtDNA-seq data of these time-course studies can reveal whether or not the transplanted mitochondria are cleared from the cells.

The RNA-seq studies are followed by proteomics analyses using a targeted approach. Due to the degeneracy of the codons, not all changes at the mRNA level are translated. Besides, proteomics analysis are conducted to capture the post-translational modifications. Targeted mass spectrometry is performed based on the differential genes identified from RNA-seq studies and the pathways involved. For example, the targeted proteins are those involved in mitochondrial retrograde and anterograde signaling, inter-mitochondrial signaling, and overall mitochondrial dynamics, as outlined below.

Proteins involved in mitochondrial retrograde and anterograde signaling are mainly calcium-dependent kinases and phosphatases, including PKC, CamKIV, JNK, MAPK, Calcineurin, and ATF2, CREB, CEBP (da Cunha F M, Torelli N Q, Kowaltowski A J. Mitochondrial retrograde signaling: Triggers, pathways, and outcomes. Oxid Med Cell Longev. 2015; 2015:482582-482582). Proteins engaged in inter-mitochondria dynamics include: (1) Drp1, Mff, Fis1, Bc1-2 family, mainly involved in fission; (2) Mfn1, Mfn2, Opal, Bc1-2 family are involved in fusion; (3) Pgc-1α, Pgc-1β, PPARγ, NRF1, NRF2, ERR α, ERR β, ERR γ are relevant to biogenesis; (4) Pink, Parkin, BNip3, Nix, Rheb, Bcl-2 family are involved in mitophagy and finally, (5) Opa-1 cleavage, cristae re-modeling, Bx/Bak channel formation and Cytochrome c release are suspected to be involved in apoptosis (Boland M L, Chourasia A H, Macleod K F. Mitochondrial dysfunction in cancer. Front Oncol. 2013; 3:292-292).

Proteins are extracted post-transplantation using Radioimmunoprecipitation assay (RIPA) buffer (Sigma-Aldrich). A protease is used to snip the protein into the peptides at defined amino acid residues. Using liquid chromatography, the peptides re separated, and the fractions are analyzed by electrospray ionization coupled to mass spectrometry, which measures and reports the peptide ions' mass-to-charge (m/z) ratio (Doerr A. Mass spectrometry-based targeted proteomics. Nature Methods. 2013; 10:23-23).

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

All figures, tables, and appendices, as well as patents, applications, and publications, referred to above, are hereby incorporated by reference.

Some embodiments have been described in connection with the accompanying drawing. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations in the claims are to be interpreted broadly based on the language employed in the claims and not limited to the examples described in the present specification or during the prosecution of the application, which examples are to be construed as non-exclusive.

What is claimed is:

1. A method of altering energy metabolism in a recipient cell comprising:
identifying the recipient cell as being in need of altering its oxidative phosphorylation status,
obtaining exogenous mitochondria, and
introducing into the recipient cell the exogenously obtained mitochondria,
wherein the exogenously obtained mitochondria functions in the recipient cell to increase or decrease oxidative phosphorylation and/or glycolysis.

2. The method of claim 1, wherein the exogenously obtained mitochondria are autologous compared to the recipient cell.

3. The method of claim 1, wherein the exogenously obtained mitochondria are non-autologous compared to the recipient cell.

4. The method of claim 1, wherein the exogenously obtained mitochondria are obtained from a xenogeneic source.

5. The method according to claim 1, wherein the recipient cell was previously subjected to ischemia or is in an individual suffering from a mitochondrial dysfunction.

6. The method according to claim 5, wherein the mitochondrial dysfunction is a condition selected from the group consisting of diabetes; a neurodegenerative disease, a neuromuscular disease, a metabolic disease, Huntington's disease; cancer; Alzheimer's disease; Parkinson's disease; amyotrophic lateral sclerosis; bipolar disorder; schizophrenia; aging; senescence; an anxiety disorder; a cardiovascular disease; sarcopenia; chronic fatigue syndrome; Leigh syndrome; Mitochondrial myopathy; Leber's hereditary optic neuropathy; Mitochondrial DNA depletion syndrome; Myoneurogenic gastrointestinal encephalopathy;
and Mitochondrial Encephalopathy, Lactic acidosis, and Stroke-like episodes (MELAS) syndrome.

7. The method of claim 1, wherein the recipient cell is located within a tissue in a subject.

8. The method of claim 7, wherein the tissue is myocardium and the recipient cell is a cardiomyocyte.

9. The method of claim 1, wherein the recipient cell is an immune cell.

10. The method of claim 9, wherein the immune cell is selected from the group consisting of a T-cell, a B-cell, a monocyte and a natural killer (NK) cell.

11. The method according to claim 1, wherein an increase in oxidative phosphorylation is accompanied by one or more of increased basal respiration, increased maximal respiration, increased coupling efficiency, reduced reactive oxygen species generation, enhanced ATP production, increased spare respiration capacity, and reduced proton leak.

12. A method of killing a recipient cell or diminishing oxidative phosphorylation and/or glycolysis in a recipient cell comprising:
obtaining an exogenous mitochondria that is defective or less efficient with regard to energy metabolism, and
introducing into the recipient cell the exogenously obtained mitochondria,
wherein the exogenously obtained mitochondria functions in the recipient cell to reduce energy metabolism.

13. The method of claim 12, wherein the recipient cell is neoplastic.

14. An isolated cell that comprises an exogenous mitochondria, wherein the cell demonstrates increased energy metabolism compared to a control cell of the same type but wherein the control cell lacks exogenously added mitochondria.

15. The isolated cell according to claim 14, wherein the cell is selected from the group consisting of a muscle cell, an immune cell, a stem cell, and a progenitor cell.

16. A method of treating a subject suffering from ischemia or a mitochondrial dysfunction comprising administering one or more group of isolated cells comprising exogenous mitochondria according to claim 14 to the subject, wherein the one or more isolated cell comprising exogenous mitochondria improve symptoms of the ischemia or the mitochondrial dysfunction.

17. A method of enhancing the cellular activity of a recipient group of cells comprising:
identifying at least one recipient cell;
obtaining at least one mitochondrion from a donor;
contacting, directly or indirectly, the donor mitochondria with the recipient cell(s) for a period of time sufficient for the recipient cell to uptake the mitochondria, thereby generating a modified recipient cell;
wherein the modified recipient cell exhibits improved energy metabolism represented by one or more of the following characteristics:
(i) increased basal respiration,
(ii) increased maximal respiration,
(iii) increased coupling efficiency,
(ii) reduced reactive oxygen species generation,
(iii) enhanced ATP production efficiency,
(iv) increased spare respiration capacity, and
(v) reduced proton leak, and
wherein the modified recipient cell exhibiting one or more of said characteristics is indicative of enhanced cellular activity.

18. The method of claim 17, wherein the recipient cell is an immune cell or a stem cell.

19. A method of for reducing or eliminating the cellular activity of a recipient cell comprising:
identifying at least one recipient cell;
obtaining at least one mitochondrion from a donor, wherein the mitochondria comprise at least a partial defect in mitochondrial function;
contacting, directly or indirectly, the donor mitochondria with the recipient cell for a period of time sufficient for the recipient cell to uptake the mitochondria, thereby generating a modified recipient cell;
wherein the modified recipient cell exhibits one or more of the following characteristics:
(i) decreased basal respiration,
(ii) decreased maximal respiration,
(iii) decreased coupling efficiency,
(iv) increased reactive oxygen species generation,
(v) reduced ATP production efficiency,
(vi) reduced spare respiration capacity, and
(vii) increased proton leak, and
(viii) Apoptosis, and
wherein the modified recipient cell exhibiting one or more of said characteristics is indicative of reduced cellular activity.

20. The method of claim 19, wherein the recipient cell is a neoplastic cell, an immune cell or a stem cell.
